SEQUENCE LISTING (13 sequences)

SEQ ID NO: 1 - protein, 10 aa, Artificial Sequence
  BDC.2.5mi
HHPIWARMDA

SEQ ID NO: 2 - protein, 15 aa, Artificial Sequence
  TOPO (722-736)
KLNYLDPRITVAWCK

SEQ ID NO: 3 - protein, 15 aa, Artificial Sequence
  ApoB (3501-3516)
SQEYSGSVANEANVY

SEQ ID NO: 4 - protein, 15 aa, Artificial Sequence
  mDSG3 (301-315)
RNKAEFHQSVISQYR

SEQ ID NO: 5 - protein, 13 aa, Artificial Sequence
  IGRP(13-25)
QHLQKDYRAYYTF

SEQ ID NO: 6 - protein, 13 aa, Artificial Sequence
  IGRP(13-25) cystrap
QHLQKDYRAYYTC

SEQ ID NO: 7 - protein, 15 aa, Artificial Sequence
  PPI (76-90) 88S cystrap
SLQPLALEGSLQSRC

SEQ ID NO: 8 - protein, 15 aa, Artificial Sequence
  PPI (76-90) 88S
SLQPLALEGSLQSRG

SEQ ID NO: 9 - protein, 13 aa, Artificial Sequence
  IGRP (23-35)
YTFLNFMSNVGDP

SEQ ID NO: 10 - protein, 13 aa, Artificial Sequence
  IGRP (23-35)
YTFLNFMSNVGDC

SEQ ID NO: 11 - protein, 12 aa, Artificial Sequence
  Glia (57-68) Cystrap
QPFPQPELPYGC

SEQ ID NO: 12 - protein, 11 aa, Artificial Sequence
  Glia (62-72) cystrap
PQPELPYPQPC

SEQ ID NO: 13 - protein, 11 aa, Artificial Sequence
  Glia (62-72)
PQPELPYPQPE

(19) United States
(12) Patent Application Publication
(10) Pub. No.: US 2022/0387583 A1
(43) Pub. Date: Dec. 8, 2022

(54) RECOMBINANT PMHC MOLECULES

(71) Applicant: UTI Limited Partnership, Calgary (CA)

(72) Inventor: Pedro SANTAMARIA, Calgary (CA)

(21) Appl. No.: 17/651,664

(22) Filed: Feb. 18, 2022

Related U.S. Application Data
(63) Continuation of application No. PCT/US2020/047191, filed on Aug. 20, 2020.
(60) Provisional application No. 62/889,734, filed on Aug. 21, 2019.

(51) Int. Cl. A61K 39/385; C07K 14/74; A61K 47/69

(52) U.S. Cl. CPC A61K 39/385; C07K 14/70539; A61K 47/6929; A61K 2039/605

(57) ABSTRACT

Provided are peptide-MHC class I and class II molecules having improved stability and high potency, and that can be produced in high yield. Also provided are receptor-signaling nanoparticles comprising the improved peptide-MHC molecules.

Assembly of soluble peptide-major histocompatibility complex class II (pMHCII) monomers into multimeric structures enables the detection of antigen-specific CD4+ T-cells in biological samples (1) and, when coated at high densities onto nanoparticles, the induction of autoantigen-specific regulatory T-cell responses capable of reversing organ-specific autoimmunity (2-4). Substitution of the transmembrane and cytoplasmic regions of the MHCII α and β chains by the c-fos and c-jun leucine zipper domains has enabled the expression of many, but not all, pMHCII molecules for diagnostic applications (5). These molecules typically are expressed at low yields and cannot be efficiently purified without the use of foreign affinity tags, which precludes their use for therapeutic applications in humans.

Provided are peptide-tethered and non-peptide-tethered MHC Class I and Class II monomers having high stability and receptor-signaling activity, and peptide-tethered and non-peptide-tethered MHC Class I and Class II monomers for reagent use. Also provided are methods for producing the peptide-tethered and non-peptide-tethered MHC Class I and Class II monomers at high yields. Also provided are therapeutic nanoparticles comprising the pMHC Class I and Class II monomers.

Also provided is an isolated pMHC monomer, wherein the pMHC monomer is a pMHC class II monomer comprising a first polypeptide and a second polypeptide, wherein: the first polypeptide and the second polypeptide meet at an interface, wherein the interface of the first polypeptide comprises an engineered protuberance which is positionable in an engineered cavity in the interface of the second polypeptide; and wherein (i) the first polypeptide comprises an MHC class II α1 domain, an MHC class II α2 domain, or a combination thereof, a first antibody C_{H}2 domain, and a first antibody C_{H}3 domain; and the second polypeptide comprises an MHC class II β1 domain, an MHC class II β2 domain, or a combination thereof, a second antibody C_{H}2 domain, and a second antibody C_{H}3 domain; wherein a disease-relevant antigen is connected to the MHC class II α1 domain or the MHC class II β1 domain by a flexible linker; or wherein (ii) the first polypeptide comprises an MHC class II β1 domain, an MHC class II β2 domain, or a combination thereof, a first antibody C_{H}2 domain, and a first antibody C_{H}3 domain, and the second polypeptide comprises an MHC class II α1 domain, an MHC class II α2 domain, or a combination thereof, a second antibody C_{H}2 domain, and a second antibody C_{H}3 domain; wherein a disease-relevant antigen is connected to the MHC class II α1 domain or the MHC class II β1 domain by a flexible linker; wherein in (i) or (ii) the engineered protuberance of the first polypeptide is in the first C_{H}3 domain, and the engineered cavity of the second polypeptide is in the second C_{H}3 domain.

In some related embodiments, the pMHC monomer is a pMHC class II monomer and the disease-relevant antigen is covalently connected to the MHC class II α1 domain or the MHC class II β1 domain by a disulfide bond formed between a cysteine amino acid associated with the antigenic peptide and a cysteine amino acid of the MHC class II α1 domain or the MHC class II β1 domain, thereby forming a cys-trapped pMHC class II monomer.

Also provided is an isolated pMHC monomer, wherein the pMHC monomer is a pMHC class I monomer comprising a first polypeptide and a second polypeptide, wherein: the first polypeptide and the second polypeptide meet at an interface, wherein the interface of the first polypeptide comprises an engineered protuberance which is positionable in an engineered cavity in the interface of the second polypeptide; and wherein (i) the first polypeptide comprises a β2-microglobulin domain, an MHC class I α1 domain, an MHC class I α2 domain, an MHC class I α3 domain, a first antibody C_{H}2 domain, and a first antibody C_{H}3 domain, and the second polypeptide comprises a second antibody C_{H}2 domain, and a second antibody C_{H}3 domain; wherein a disease-relevant antigen is connected to the β2-microglobulin domain by a flexible linker; or wherein (ii) the first polypeptide comprises a first antibody C_{H}2 domain, and a first antibody C_{H}3 domain, and the second polypeptide comprises a β2-microglobulin domain, an MHC class I α1 domain, an MHC class I α2 domain, an MHC class I α3 domain, a second antibody C_{H}2 domain, and a second antibody C_{H}3 domain; wherein a disease-relevant antigen is connected to the β2-microglobulin domain by a flexible linker; wherein in (i) or (ii) the engineered protuberance of the first polypeptide is in the first C_{H}3 domain, and the engineered cavity of the second polypeptide is in the second C_{H}3 domain.

In some related embodiments, the pMHC monomer is a pMHC class I monomer and the second polypeptide of (i) or the first polypeptide of (ii) further comprises, N-terminal to the second antibody C_{H}2 domain, a β2-microglobulin domain, an MHC class I α1 domain, an MHC class I α2 domain, and an MHC class I α3 domain. In some embodiments, the pMHC monomer is a pMHC class I monomer and wherein the disease-relevant antigen is covalently connected to the MHC class I α1 domain and/or the MHC class I α2 domain by at least one disulfide bond formed between a cysteine amino acid associated with the antigenic peptide and a cysteine amino acid of the MHC class I α1 domain and/or the MHC class I α2 domain, thereby forming a cys-trapped pMHC class I monomer.

In some embodiments, the isolated pMHC class I or class II monomer does not comprise an affinity-purification tag. In some embodiments, the disease-relevant antigen is an autoimmune-disease-relevant antigen.

In some embodiments wherein the pMHC monomer is a pMHC class II monomer, the cysteine amino acid is within 10 amino acids of a residue that forms a part of an MHC binding groove. In some related embodiments, the cysteine amino acid is within 3 amino acids of a residue that forms a part of the MHC binding groove. In some related embodiments, the cysteine amino acid of the MHC class II ≢1 domain or the MHC class II β1 domain has been introduced into the naturally occurring sequence of the MHC class II α1 domain or the MHC class II β1 domain.

In some embodiments wherein the pMHC monomer is a pMHC class I monomer, the cysteine amino acid of the MHC class I α1 domain and/or the MHC class I α2 domain has been introduced into the naturally occurring sequence of the MHC class II α1 domain or the MHC class II β1 domain.

Also provided is the isolated pMHC monomer as set forth, for use in treating an individual diagnosed with or suspected of being afflicted with an autoimmune disease. In some embodiments, the polynucleotide encodes the first or the second polypeptide. In some embodiments, the host cell comprises the polynucleotide. In some embodiments, the polynucleotide is stably integrated into the genome of the host cell. In some embodiments, the isolated pMHC monomer is conjugated to a nanoparticle to form a pMHC monomer-nanoparticle conjugate, wherein the nanoparticle is non-liposomal, has a solid core, or both. In some embodiments, the solid core is a gold, iron, or iron oxide core. In some embodiments, the solid core has a diameter of less than 100 nanometers. In some embodiments, the at least one isolated pMHC monomer is covalently linked to the nanoparticle. In some embodiments, the at least one pMHC monomer is covalently linked to the nanoparticle through a linker comprising polyethylene glycol (PEG). In some embodiments, the polyethylene glycol is functionalized with maleimide. In some embodiments, the polyethylene glycol is less than 5 kD.

Also provided is a pharmaceutical composition comprising the pMHC monomer-nanoparticle conjugate described herein, and a pharmaceutical excipient, stabilizer, or diluent. Also provided is the pMHC monomer-nanoparticle conjugate as set forth, or the pharmaceutical composition as set forth, for use in a method of treating an autoimmune disease or inflammatory condition.

Also provided is a method of treating an autoimmune disease or inflammatory condition comprising administering to an individual an isolated pMHC monomer-nanoparticle conjugate or the pharmaceutical composition as set forth.

Also provided is a method for production and purification of the isolated pMHC monomer described herein, comprising the steps of: culturing a host cell comprising a nucleic acid encoding the first and second polypeptide; and a) purifying the pMHC monomer from the host cell culture; or the steps of: a) culturing a first host cell comprising a nucleic acid encoding the first polypeptide; b) culturing a second host cell comprising a nucleic acid encoding the second polypeptide; c) purifying the polypeptides from the first and second host cell cultures; and d) forming the purified pMHC monomer by incubating the first and second polypeptides together. In some embodiments, none of the nucleic acids encode an affinity-purification tag. In some embodiments, the purifying comprises applying a liquid comprising the pMHC monomer or the polypeptides to a liquid chromatography column. In some embodiments, the liquid chromatography column comprises Protein A, Protein G, or both. In some embodiments, method for production and purification of the isolated pMHC monomer further comprises measuring the yield of the purified pMHC monomer. In some embodiments, the purified pMHC monomer is a cys-trapped pMHC monomer. In some embodiments, the measured yield of the purified cys-trapped pMHC monomer is about 10 to about 30 times greater than that of a comparable non-cys-trapped conventional leucine-zippered pMHC monomer, respectively.

Also provided is a high potency receptor-signaling pMHC monomer-nanoparticle conjugate, comprising a nanoparticle core coupled to a plurality of isolated pMHC monomers as set forth herein, optionally wherein the pMHC monomers are coupled to the nanoparticle at a low valency or low density, and wherein the plurality of pMHC monomers comprises one or more pMHC monomer species, wherein each pMHC monomer species comprises a different disease-relevant antigen. In some embodiments of the high potency receptor-signaling pMHC monomer-nanoparticle conjugate, the pMHC monomers are cys-trapped pMHC monomers. In some embodiments, the low valency is a pMHC monomer to nanoparticle ratio of about 10:1 to about 50:1. In some embodiments, the low valency is a pMHC monomer to nanoparticle ratio of about 20:1 to about 30:1. In some embodiments, the receptor-signaling potency that is at least about 1.5 times greater than a comparable receptor-signaling pMHC monomer-nanoparticle conjugate that comprises non-cys-trapped pMHC monomers at the same valency or density. In some embodiments, the receptor-signaling potency that is about 1.5 to about 5 times greater than a comparable receptor-signaling pMHC monomer-nanoparticle conjugate that comprises non-cys-trapped pMHC monomers at the same valency or density. In some embodiments, the nanoparticle is non-liposomal, has a solid core, or both.

Also provided is a method for making a high potency receptor-signaling pMHC monomer-nanoparticle conjugate comprising: coupling a nanoparticle core to a plurality of isolated pMHC monomers as set forth herein, optionally wherein the pMHC monomers are coupled to the nanoparticle at a low valency or low density, and wherein the plurality of pMHC monomers comprises one or more pMHC monomer species, wherein each pMHC monomer species comprises a different disease-relevant antigen. In some embodiments, the pMHC monomers are cys-trapped pMHC monomers. In some embodiments, the low valency is a pMHC monomer to nanoparticle ratio of about 10:1 to about 50:1. In some embodiments, the low valency is a pMHC monomer to nanoparticle ratio of about 20:1 to about 30:1. In some embodiments, the nanoparticle is non-liposomal, has a solid core, or both. In some embodiments, the solid core is a gold, iron, or iron oxide core. In some embodiments, the solid core has a diameter of less than 100 nanometers. In some embodiments, the at least one isolated pMHC monomer is covalently linked to the nanoparticle. In some embodiments, the at least one pMHC monomer is covalently linked to the nanoparticle through a linker comprising polyethylene glycol (PEG). In some embodiments, the polyethylene glycol is functionalized with maleimide. In some embodiments, the polyethylene glycol is less than 5 kD. In some embodiments, the method for making a high potency receptor-signaling pMHC monomer-nanoparticle conjugate further comprises the step of measuring the receptor-signaling potency of the high potency receptor-signaling pMHC monomer-nanoparticle conjugate. In some related embodiments, the pMHC monomer of the high potency receptor-signaling pMHC monomer-nanoparticle conjugate is cys-trapped, and the measured receptor-signaling potency is at least about 1.5 times greater than a comparable receptor-signaling pMHC monomer-nanoparticle conjugate that comprises non-cys-trapped pMHC monomers at the same valency or density. In some embodiments, the measured receptor-signaling potency is about 1.5 to about 5 times greater than a comparable receptor-signaling pMHC monomer-nanoparticle conjugate that comprises non-cys-trapped pMHC monomers at the same valency or density. In some embodiments, the method for making a high potency receptor-signaling pMHC monomer-nanoparticle conjugate further comprises selecting an optimal high potency receptor-signaling cys-trapped pMHC monomer-nanoparticle conjugate for use in a therapeutic composition when the measured receptor-signaling potency is at least about 1.5 times greater than a comparable receptor-signaling pMHC monomer-nanoparticle conjugate that comprises non-cys-trapped pMHC monomers at the same valency or density. In some embodiments, the method comprises selecting high potency receptor-signaling of the high potency receptor-signaling cys-trapped pMHC monomer-nanoparticle conjugate pMHC monomer-nanoparticle for use in a therapeutic composition when the measured receptor-signaling potency is about 1.5 to about 5 times greater than comparable receptor-signaling pMHC monomer-nanoparticle conjugate that comprises non-cys-trapped pMHC monomers at the same valency or density. Also provided is a pharmaceutical composition comprising the optimal high potency pMHC receptor-signaling monomer-nanoparticle conjugate as selected, and a pharmaceutical excipient, stabilizer, or diluent. Also provided is the optimal high potency pMHC receptor-signaling monomer-nanoparticle conjugate selected as set forth or the pharmaceutical composition as set forth, for use in a method of treating an autoimmune disease or inflammatory condition. Also provided is a method of treating an autoimmune disease or inflammatory condition comprising administering to an individual the high potency pMHC receptor-signaling monomer-nanoparticle conjugate, the selected optimal high potency pMHC receptor-signaling monomer-nanoparticle conjugate, or the pharmaceutical composition.

Also provided is a method for high-yield production and purification of an MHC monomer, wherein the MHC monomer is an MHC class II monomer comprising a first polypeptide and a second polypeptide, wherein: the first polypeptide and the second polypeptide meet at an interface, wherein the interface of the first polypeptide comprises an engineered protuberance which is positionable in an engineered cavity in the interface of the second polypeptide; and (i) the first polypeptide comprises an MHC class II α1 domain, an MHC class II α2 domain, or a combination thereof, a first antibody C_{H}2 domain, and a first antibody C_{H}3 domain; and the second polypeptide comprises an MHC class II β1 domain, an MHC class II β2 domain, or a combination thereof, a second antibody C_{H}2 domain, and a second antibody C_{H}3 domain; or (ii) the first polypeptide comprises an MHC class II β1 domain, an MHC class II β2 domain, or a combination thereof, a first antibody C_{H}2 domain, and a first antibody C_{H}3 domain; and the second polypeptide comprises an MHC class II α1 domain, an MHC class II α2 domain, or a combination thereof, a second antibody C_{H}2 domain, and a second antibody C_{H}3 domain; wherein in (i) or (ii) the engineered protuberance of the first polypeptide is in the first C_{H}3 domain, and the engineered cavity of the second polypeptide is in the second C_{H}3 domain, the method comprising the steps of: a) culturing a host cell comprising a nucleic acid encoding the first and second polypeptide; and b) purifying the MHC class II monomer from the host cell culture; or the steps of: a) culturing a first host cell comprising a nucleic acid encoding the first polypeptide; b) culturing a second host cell comprising a nucleic acid encoding the second polypeptide; c) purifying the polypeptides from the first and second host cell cultures; and d) forming the MHC class II monomer by incubating the first and second polypeptides together.

Also provided is a method for high-yield production and purification of an MHC monomer, wherein the MHC monomer is an MHC class I monomer comprising a first polypeptide and a second polypeptide, wherein: the first polypeptide and the second polypeptide meet at an interface, wherein the interface of the first polypeptide comprises an engineered protuberance which is positionable in an engineered cavity in the interface of the second polypeptide; and (i) the first polypeptide comprises an MHC class I α2 domain, an MHC class I α3 domain, or a combination thereof, a first antibody C_{H}2 domain, and a first antibody C_{H}3 domain; and the second polypeptide comprises an MHC class I α1 domain, a β-microglobulin, or a combination thereof, a second antibody C_{H}2 domain, and a second antibody C_{H}3 domain; or (ii) the first polypeptide comprises an MHC class I α1 domain, a β-microglobulin domain, or a combination thereof, a first antibody C_{H}2 domain, and a first antibody C_{H}3 domain; and the second polypeptide comprises an MHC class I α2 domain, an MHC class I α3 domain, or a combination thereof, a second antibody C_{H}2 domain, and a second antibody C_{H}3 domain; wherein in (i) or (ii) the engineered protuberance of the first polypeptide is in the first C_{H}3 domain, and the engineered cavity of the second polypeptide is in the second C_{H}3 domain, the method comprising the steps of: a) culturing a host cell comprising a nucleic acid encoding the first and second polypeptide; and b) purifying the MHC class I monomer from the host cell culture; or the steps of: a) culturing a first host cell comprising a nucleic acid encoding the first polypeptide; b) culturing a second host cell comprising a nucleic acid encoding the second polypeptide; c) purifying the polypeptides from the first and second host cell cultures; and d) forming the MHC class I monomer by incubating the first and second polypeptides together. In some embodiments of these methods for high-yield production and purification of an MHC monomer, none of the nucleic acids encode an affinity-purification tag. In some embodiments, the first polypeptide and the second polypeptide of (i) and (ii) do not comprise an affinity-purification tag. In some embodiments, the purifying comprises applying a liquid comprising the MHC monomer or polypeptides to a liquid chromatography column. In some embodiments, the liquid chromatography column comprises Protein A, Protein G, or both. In some embodiments, these methods further comprise loading the purified MHC monomer in vitro with a disease-relevant peptide antigen, thereby forming a non-peptide tethered pMHC monomer. In some embodiments, the non-peptide tethered pMHC monomer is a cys-trapped non-peptide tethered pMHC monomer. In some embodiments, the disease-relevant antigen is an autoimmune-disease-relevant antigen. In some embodiments, these methods further comprise measuring the yield of the expressed pMHC monomer. In some embodiments, the pMHC monomer is a cys-trapped pMHC monomer, and wherein the measured yield of the expressed cys-trapped pMHC monomer is about 10 to about 30 times greater than that of a comparable non-cys-trapped conventional leucine-zippered pMHC monomer. Also provided is an MHC monomer produced using any method as set forth above.

In some embodiments, the MHC of the pMHC class I monomer or MHC class I monomer comprises all or part of a HLA-A, HLA-B, HLA-C, HLA-E, HLA-F, HLA-G, or CD1-like (non-classical) molecule. In some embodiments, the MHC of the pMHC class II monomer or MHC class II monomer comprises all or part of a HLA-DR, HLA-DQ, HLA-DP, HLA-DM, HLA-DOA, or HLA-DOB molecule.

Also provided is a method for making a pMHC class I or class II multimer, the method comprising multimerizing a plurality of pMHC class I or class II monomers as set forth herein, or a plurality of pMHC monomers made using the method as set forth herein. Also provided is a pMHC multimer comprising a pMHC monomer as set forth herein or made by a method described herein. In some embodiments, the pMHC multimer comprises 2 to 10 pMHC class I or class II monomers (heterodimers). In some embodiments, the pMHC multimer is a tetramer, pentamer, or dextramer. In some embodiments, the pMHC multimer is labeled. Also provided is the use of the pMHC multimer for a therapeutic application. Also provided is the use of the pMHC multimer for a diagnostic application.

Also provided is a method for making an MHC multimer, the method comprising multimerizing an MHC monomer as set forth herein, or an MHC monomer made using a method as set forth herein, wherein the MHC monomer is loaded with antigen in vitro.

Also provided is a plurality of pMHC multimers as set forth, and/or MHC multimers made by a method as set forth, wherein the plurality of pMHC multimers and/or MHC multimers comprises one or more pMHC monomer species and/or one or more MHC monomer species, wherein each pMHC monomer species and/or MHC monomer species comprises a different disease-relevant antigen. In some embodiments, the plurality of pMHC multimers and/or MHC multimers comprises 2 to 500 pMHC monomer species and/or MHC monomer species, wherein each pMHC monomer species and/or MHC monomer species comprises a different disease-relevant antigen.

Also provided is a plurality of high potency receptor-signaling pMHC monomer-nanoparticle conjugates as set forth herein. In some embodiments, the plurality of pMHC monomers of the pMHC monomer-nanoparticle conjugates comprises 2 to 500 pMHC monomer species, wherein each pMHC monomer species comprises a different disease-relevant antigen.

Also provided is a high potency receptor-signaling MHC monomer-nanoparticle conjugate, comprising a nanoparticle core coupled to a plurality of isolated non-peptide tethered pMHC monomers as set forth, optionally wherein the non-peptide tethered pMHC monomers are coupled to the nanoparticle at a low valency or low density, and wherein the plurality of non-peptide tethered pMHC monomers comprises one or more non-peptide tethered pMHC monomer species, wherein each non-peptide tethered pMHC monomer species comprises a different disease-relevant antigen. In some embodiments, the non-peptide tethered pMHC monomers are cys-trapped non-peptide tethered pMHC monomers. In some embodiments, the low valency is a non-peptide tethered pMHC monomer to nanoparticle ratio of about 10:1 to about 50:1. In some embodiments, the low valency is a non-peptide tethered pMHC monomer to nanoparticle ratio of about 20:1 to about 30:1. In some embodiments, the nanoparticle is non-liposomal, has a solid core, or both.

Also provided is a method for making a high potency receptor-signaling non-peptide tethered pMHC monomer-nanoparticle conjugate comprising: coupling a nanoparticle core to a plurality of isolated non-peptide tethered pMHC monomers as set forth herein, optionally wherein the pMHC monomers are coupled to the nanoparticle at a low valency or low density, and wherein the plurality of non-peptide tethered pMHC monomers comprises one or more non-peptide tethered pMHC monomer species, wherein each non-peptide tethered pMHC monomer species comprises a different disease-relevant antigen. In some embodiments, the non-peptide tethered pMHC monomers are cys-trapped non-peptide tethered pMHC monomers. In some embodiments, the low valency is a non-peptide tethered pMHC monomer to nanoparticle ratio of about 10:1 to about 50:1. In some embodiments, the low valency is a non-peptide tethered pMHC monomer to nanoparticle ratio of about 20:1 to about 30:1. In some embodiments, the nanoparticle is non-liposomal, has a solid core, or both. In some embodiments, the solid core is a gold, iron, or iron oxide core. In some embodiments, the solid core has a diameter of less than 100 nanometers. In some embodiments, the at least one isolated non-peptide tethered pMHC monomer is covalently linked to the nanoparticle. In some embodiments, the at least one non-peptide tethered pMHC monomer is covalently linked to the nanoparticle through a linker comprising polyethylene glycol (PEG). In some embodiments, the polyethylene glycol is functionalized with maleimide. In some embodiments, the polyethylene glycol is less than 5 kD.

Also provided is a pharmaceutical composition comprising a high potency non-peptide tethered pMHC receptor-signaling monomer-nanoparticle conjugate as set forth herein, and a pharmaceutical excipient, stabilizer, or diluent. Also provided is a high potency non-peptide tethered pMHC receptor-signaling monomer-nanoparticle conjugate or the pharmaceutical composition is used in a method of treating an autoimmune disease or inflammatory condition.

Also provided is a method of treating an autoimmune disease or inflammatory condition comprising administering to an individual the high potency non-peptide tethered pMHC receptor-signaling monomer-nanoparticle conjugate as set forth herein, or the pharmaceutical composition as set forth herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments.

FIGS. 1A-1C. Construct structure. Cartoons depict the general structure of the lentiviral system (FIG. 1A) and the type of constructs used (FIG. 1B and FIG. 1C). FIG. 1B. Structure of a P2A-linked pMHCβ and MHCα chain-coding construct (top) and a representation of the resulting pMHCII product secreted into the cell culture supernatant (bottom). FIG. 1C. Single pMHCβ or MHCα chain constructs that were serially transduced into CHO cells to produce the resulting pMHCIIαβ heterodimers (bottom).

FIGS. 2A-2D. Key junctional, linker and motif sequences for the various constructs used herein. FIG. 2A. Key amino acid sequences of human pMHCII molecules encoding a cys-trapped IGRP₁₃₋₂₅/DRA1*0101/DRB1*0301 pMHCII heterodimerized via c-jun/c-fos leucine zippers (also referred to “conventional”). “. . . ” are used to indicate that the corresponding intervening amino acid sequences are not shown, as they are publicly available. Residues in red are mutated and the original residue and its position are indicated immediately below. FIG. 2B. Key amino acid sequences of a murine pMHCII molecule encoding BDC2.5mi/IAα^{d}/IAβ^{g7} heterodimerized using a carboxyterminal murine IgG1-Fc based KIH. FIG. 2C. Key amino acid sequences of a murine pMHCII molecule encoding BDC2.5mi/IAα^{d}/IAβ^{g7} heterodimerized using a carboxyterminal human IgG1-Fc based KIH. FIG. 2D. Key amino acid sequences for “empty” human MHCII molecules encoding DRA*0101 MHCα and DRB1, DRB3, DRB4 or DRB5 MHCβ chains heterodimerized using a carboxyterminal human IgG1-Fc based KIH.

FIG. 3. Cartoons depicting the structures of the KIH-based pMHCII constructs or specific domains. Left, primary structure of a Cys-trapped KIH-based pMHCII heterodimer. Top right, secondary structure of the peptide-binding domain loaded with a peptide bound to the MHCII molecule on a specific register via a disulphide bridge between the carboxyterminal end of the peptide and a complementary Cys on the MHCIIα chain. Bottom right, predicted quaternary structure of the KIH Fc portion of the KIH-based constructs and the key amino acid substitutions that were used to promote KIH-based heterodimerization.

FIGS. 4A-4C. Stabilization of pMHCII heterodimers by introduction of peptide-MHCα chain Cys-traps. FIG. 4A. SDS-PAGE for various c-jun/c-fos-based pMHCII heterodimers carrying or lacking Cys-traps (CT), under native vs. denaturing conditions. Lane 1, BDC2.5mi/IA^{g7}; Lane 2, IGRP₁₃₋₂₅/DR3; Lane 3, PPI_{(76-90)(88S)}/DR4; 4, IGRP₂₃₋₃₅/DR4; 5, Topo₇₂₂₋₇₃₆/IA^{b}; 6, ApoB₃₅₀₁₋₃₅₁₆/IA^{b}; 7, DSG3₃₀₁₋₃₁₅/IAb. MW, Molecular Weight markers. Except for ApoB₃₅₀₁₋₃₅₁₆/IA^{b}, all other pMHCII heterodimers shown are partially or completely SDS unstable. FIG. 4B. Effects of Cys-trapping on SDS stability of pMHCII heterodimers. Data correspond to: Lane 1, IGRP₁₃₋₂₅/DR3-non-CT; Lane 2, IGRP₁₃₋₂₅/DR3-CT; Lane 3, IGRP₂₃₋₃₅/DR4-non-CT; Lane 4, IGRP₂₃₋₃₅/DR4-CT; Lane 5, PPI_{76-90(88S)}/DR4-non-CT; and Lane 6, Glia₆₂₋₇₂/DQ2-CT. FIG. 4C. Representative pMHCII tetramer/CD4 FACS dot plots for Jurkat cells expressing human CD4 and an IGRP₁₃₋₂₅/DR3-specific TCR (top) or mouse CD4 and a BDC2.5mi/IAg^{g7}-specific TCR (bottom) stained with non-CT (left) or CT (right) IGRP₁₃₋₂₅/DR3 tetramers.

FIGS. 5A-5E. Introduction of a c-jun/c-fos leucine zipper into a KIH-based pMHCII is incompatible with formation and secretion of pMHCII heterodimers. FIG. 5A and 5B show cartoons displaying the structure of the two types of KIH constructs tested. FIG. 5C shows expression of eGFP in CHO-S cell lines transduced with lentiviruses encoding the constructs depicted in FIG. 5A (upper left graph) and FIG. 5B (lower left graph), indicating adequate construct transcription and translation. FIG. 5C shows FPLC elution profiles of pMHC class II from Strep-tactin columns loaded with supernatants from CHO cells expressing the constructs in FIG. 5A (upper right graph) or FIG. 5B (lower right graph). Note the absence of any detectable pMHC in the supernatants from the former. FIG. 5D shows effects of the KIH on the SDS stability of a representative pMHCII heterodimer, in the absence of Cys-trapping. Data correspond to c-jun/c-fos-based BDC2.5mi/IA^{g7}(‘conv’, left lane) and a KIH-based BDC2.5mi/IA^{g7}(right lane). FIG. 5E shows representative pMHCII tetramer/eGFP (TCR) FACS dot plots for BDC2.5-TCR-transgenic CD4+ T-cells stained with c-jun/c-fos-(‘conv’, left plot) or KIH-based BDC2.5mi/IA^{g7} tetramers (right plot).

FIGS. 6A-6E. Nanoparticles coated with representative KIH-based pMHCII monomers have similar potency and in vivo biological activity as those carrying c-jun/c-fos-based monomers. FIG. 6A shows native (left panel) and denaturing (right panel) SDS-PAGE for NPs coated with a representative KIH-based pMHCII molecule. PFM denotes the iron oxide NP. Legend: MW: molecular weight markers; 1: 2 μg of KIH-based BDC2.5mi/IA^{g7} monomers; 2: 2.2 μL of PFM coated with KIH-based BDC2.5mi/IA^{g7} monomers; 3: 1.1 μL of PFM coated with KIH-based BDC2.5mi/IA^{g7} monomers; 4: 2 μg of KIH-based BDC2.5mi/IA^{g7} monomers; 5: 2.2 μL of PFM coated with KIH-based BDC2.5mi/IA^{g7} monomers; 6: 1.1 μL of PFM coated with KIH-based BDC2.5mi/IA^{g7} monomers. FIG. 6B shows luciferase activity induced by NPs coated with c-jun/c-fos- (‘conv’) or KIH-based BDC2.5mi/IA^{g7} monomers (normalized to that induced by soluble anti-CD3ε mAb) on Jurkat cells co-expressing mouse CD4, a BDC2.5mi/IA^{g7}-specific TCR and an NFAT-driven luciferase reporter. Data correspond to mean+SEM of triplicates. FIG. 6C shows percentages of BDC2.5mi/IA^{g7} tetramer-positive CD4+ T-cells in blood, spleen, pancreatic lymph nodes (PLN), mesenteric lymph nodes (MLN) and bone marrow (BM) from NOD mice treated (twice a wk for 5 wks) with NPs coated with c-jun/c-fos-based (‘conv’) BDC2.5mi/IA^{g7} or KIH-based BDC2.5mi/IA^{g7} monomers (20 μg pMHC/dose). Data correspond to average+SEM values from 4 mice/group. FIG. 6D shows cytokine profile of the tetramer+ cells isolated from the mice in FIG. 6C. Tetramer+ cells were challenged with anti-CD3/anti-CD28 mAb-coated beads for 3 days and the supernatants assayed for cytokine content using Luminex technology. Data correspond to average+SEM values of cells isolated from 4 mice/group. FIG. 6E shows luciferase activity induced by NPs coated with KIH-based BDC2.5mi/IA^{g7} pMHCs carrying a murine or a human Fc-based KIH (normalized to that induced by soluble anti-CD3ε mAb) on Jurkat cells co-expressing mouse CD4, a BDC2.5mi/IA^{g7}-specific TCR and an NFAT-driven luciferase reporter. Data correspond to mean+SEM of triplicates.

FIGS. 7A-7D. FIG. 7A and FIG. 7B show luciferase activity induced by NPs coated with c-jun/c-fos- (‘conv’) or KIH-based BDC2.5mi/IA^{g7} monomers. Activity was normalized to that induced by soluble anti-CDE mAb) on Jurkat cells co-expressing mouse CD4, a BDC2.5mi/IA^{g7}-specific TCR and an NFAT-driven luciferase reporter. Data correspond to FIG. 6B but normalized by molar concentration of pMHCII or NP number. FIG. 7C and FIG. 7D show luciferase activity induced by NPs coated with KIH-based BDC2.5mi/IA^{g7} pMHCIIs carrying a murine or a human Fc-based KIH (normalized to that induced by soluble anti-CD3ε mAb) on Jurkat cells co-expressing mouse CD4, a BDC2.5mi/IA^{g7}-specific TCR and an NFAT-driven luciferase reporter. Data correspond to FIG. 6E but normalized by molar concentration of pMHCII or NP number. Data correspond to mean±SEM of triplicates.

FIGS. 8A-8I. The KIH Fc enables the generation of pMHCIIs with increased biological potency and stabilizes “empty” MHCII heterodimers for expression and peptide loading. FIG. 8A shows representative pMHCII tetramer/eGFP (TCR) FACS dot plots for Jurkat cells expressing human CD4 and an IGRP₁₃₋₂₅/DR3-specific TCR or mouse CD4 and a BDC2.5mi/IA^{g7}-specific TCR (negative control) stained with c-jun/c-fos- (‘conv’) or KIH-based IGRP₁₃₋₂₅/DR3 tetramers. FIG. 8B shows representative pMHC class II tetramer/eGFP (TCR) FACS dot plots for the same Jurkat cells used in A, but stained with KIH-based IGRP₁₃₋₂₅/DR3 tetramers lacking (left) or carrying a CT (right). FIG. 8C shows introduction of a CT into KIH-based human pMHCII molecules does not alter their reactivity with a MHCII-specific mAb binding to a conformational epitope, as measured by ELISA. Data correspond to mean+SEM of triplicates. FIG. 8D shows luciferase activity induced by NPs coated with c-jun/c-fos-based (‘conv’), CT IGRP₁₃₋₂₅/DR3 pMHCs vs. NPs coated with non-CT, KIH-based IGRP₁₃₋₂₅/DR3 coated at three different valencies on Jurkat cells co-expressing human CD4, an IGRP₁₃₋₂₅/DR3-specific TCR and NFAT-driven luciferase. Data correspond to mean+SEM of triplicates. Note that use of the KIH structure cannot overcome the positive effects of CT on the potency of NPs displaying c-jun/c-fos-based pMHCIIs, which coat at higher valencies owing to their smaller size. FIG. 8E shows luciferase activity induced by NPs coated with c-jun/c-fos-based/CT (‘conv’) or KIH-based/CT IGRP₁₃₋₂₅/DR3 monomers vs. their non-CT counterparts on Jurkat cells co-expressing human CD4, an IGRP₁₃₋₂₅/DR3-specific TCR and NFAT-driven luciferase. Data correspond to mean+SEM of triplicates. Note that nanomedicines displaying KIH-based pMHCII monomers have potencies similar to those obtained with their c-jun/c-fos-based counterparts, at significantly lower valencies. Addition of a CT to these nanomedicines carrying KIH-based monomers increases their potency to levels similar to those seen with nanomedicines carrying conventional, CT pMHCIIs at higher valencies. FIG. 8F shows SDS-PAGE of CT leucine-zippered (1) or KIH-based (2) Gliadin₆₂₋₇₂/DQB1*0201/DQA1*0501 monomers. The upper band labeled as “αβx2” corresponds to non-covalent dimers of heterodimers (can be dissociated via sonication, not shown). FIG. 8G shows representative pMHCII tetramer/CD4 FACS dot plots for Jurkat cells expressing human CD4 and an IGRP₁₃₋₂₅/DR3-specific (top) or mouse CD4 and a BDC2.5mi/IA^{g7}-specific TCR (bottom) stained with c-jun/c-fos-based (‘conv’) IGRP₁₃₋₂₅/DR3 tetramer (peptide-linked; left) or tetramers generated using peptide-loaded empty DR3-KIH monomers (right). FIG. 8H shows representative pMHCII tetramer/eGFP (TCR) FACS dot plots for Jurkat cells expressing human CD4 and PDC-E2₁₂₂₋₃₅/DRB4-specific TCR (top left), a PDC-E2₂₄₉₋₂₆₂/DRB4-specific TCR (top right) or a IGRP₁₃₋₂₅/DR3-specific TCR (bottom panels; negative control). Cells were stained with PDC-E2₁₂₂₋₁₃₅/DRB4 or PDC-E2₂₄₉₋₂₆₂/DRB4-tetramers carrying linker-tethered or exogenously loaded peptides. FIG. 8I shows signal amplification of KIH-based tetramer binding using anti-human Fc antibodies. Human PBMCs (10⁶) were spiked with cells from a human IGRP₁₃₋₂₅/DR3-specific T-cell clone (top row) or with an irrelevant (PPI_{(76-90(88S)}/DR4-specific) T-cell clone (10⁴) (bottom row). Cells were treated with the protein kinase inhibitor Dasatinib (right panel) or left untreated (left panel) and then stained with PE-labeled KIH-based IGRP1₁₃₋₂₅/DR3 tetramers. Tetramer staining was amplified with PE-labeled anti-IgG antibodies. Values on the plots correspond to the geometric mean fluorescence intensity for pMHC tetramer staining.

FIGS. 9A-9D. FIG. 9A and FIG. 9B show luciferase activity induced by NPs coated with c-jun/c-fos-based (‘conv’), Cys-trapped IGRP₁₃₋₂₅/DR3 pMHCs vs. NPs coated with non-Cys-trapped KIH-based IGRP₁₃₋₂₅/DR3 coated at three different valencies on Jurkat cells co-expressing human CD4, an IGRP₁₃₋₂₅/DR3-specific TCR and an NFAT-driven luciferase reporter. Data correspond to FIG. 8D but normalized by molar concentration of pMHCII or NP number. FIG. 9C and FIG. 9D show luciferase activity induced by NPs coated with c-jun/c-fos-based/Cys-trapped or KIH-based/Cys-trapped IGRP₁₃₋₂₅/DR3 monomers vs. their non-Cys-trapped counterparts on Jurkat cells co-expressing human CD4, an IGRP₁₃₋₂₅/DR3-specific TCR and an NFAT-driven luciferase reporter. Data correspond to FIG. 8E but normalized by molar concentration of pMHCII or NP number. Data correspond to mean+SEM of triplicates.

### DETAILED DESCRIPTION

Soluble MHC Molecules

Provided are constructs and methods for producing soluble peptide-tethered MHC Class I or Class II monomers (also referred to herein as pMHC Class I and pMHC Class II monomers, pMHC Class I and pMHC Class II heterodimers, pMHCI monomers and pMHC II monomers, pMHC Class I and pMHC Class II molecules, and pMHCI heterodimers and pMHCII heterodimers). Also provided are constructs and methods for the production of soluble non-peptide-tethered (“empty”) MHC Class I or Class II monomers (also referred to herein as MHC Class I and MHC Class II monomers, MHC Class I and MHC Class II heterodimers, MHCI monomers and MHCII monomers, MHCI molecules and MHC II molecules, and MHCI heterodimers and MHCII heterodimers) that can be loaded in vitro with antigen to form non-peptide-tethered molecules (non-peptide tethered pMHC Class I and pMHC Class II monomers, non-peptide tethered pMHC Class I and pMHC Class II heterodimers, non-peptide tethered pMHCI monomers and pMHC II monomers, and non-peptide tethered pMHCI heterodimers and pMHCII heterodimers). These molecules are highly stable and are useful as monomers, e.g., soluble, or bound in substrate conjugates, or in multimers, e.g., for T-cell therapeutic and diagnostic applications. Uses include in vivo targeting and regulation of T cells involved in autoimmune disease, and detection and isolation of antigen-specific T-cells. The peptide-tethered or non-peptide tethered pMHC Class I or Class II monomers can be used to generate multimer complexes, e.g., labeled pMHC Class I or Class II tetramers, or larger multimers. In some embodiments, pMHC or MHC Class I or Class II molecules are used in therapeutic or diagnostic methods, e.g., as tools for characterizing T cell specificity and phenotype. In these embodiments the pMHC Class I or Class II molecules may comprise a loaded antigen (referred to as non-peptide tethered pMHC molecules). In some embodiments, the pMHC or MHC monomers are bound to a substrate, e.g., a nanoparticle. In some embodiments, the compositions and methods are used to prepare pMHC and/or MHC monomers that are multimerized for reagent use, e.g., for T-cell isolation and detection. In some embodiments, pMHC (peptide-tethered and non-peptide tethered) and/or MHC multimers are used for T-cell diagnostics. In some embodiments, pMHC (peptide-tethered and non-peptide tethered) and/or MHC multimers are used for therapeutic purposes. Uses for pMHC (peptide-tethered and non-peptide tethered) or MHC multimers assembled using stable pMHC or MHC monomers include those described in the literature, e.g., by Bakker et al., “MHC Multimer Technology: Current Status and Future Prospects,” Current Opinion in Immunology, 17(4):428-433, 2005, and by Nepom, G., 2012, “MHC Class II Tetramers,” J. Immunology 188(6): 2477-2482 both incorporated herein by reference. In some embodiments, an MHC Class I monomer (peptide-tethered, non-peptide tethered, or empty) comprises all or part of a HLA-A, HLA-B, HLA-C, HLA-E, HLA-F, HLA-G molecule. In some embodiments, an MHC class I monomer (peptide-tethered, non-peptide tethered, or empty) comprises all or part of a non-classical molecule. In some embodiments the non-classical molecule is a CD1-like molecule. In some embodiments, an MHC Class II monomer (peptide-tethered, non-peptide tethered, or empty) comprises all or part of a HLA-DR, HLA-DQ, HLA-DP, HLA-DM, HLA-DOA, or HLA-DOB molecule.

As described herein, fusion of peptide-tethered (also referred to herein as “antigen-stabilized”) or empty (“non-antigen-stabilized” or “non-peptide tethered”) MHCII αβ chains to the IgG1-Fc mutated to form knob-into-hole (KIH) structures results in the assembly of highly stable (p)MHCII monomers. The designs described herein allow the expression and rapid purification of challenging pMHCII types at high yields without the need to use leucine zippers or purification affinity tags. These designs increase the antigen-receptor signaling potency of multimerized derivatives useful for therapeutic applications, and facilitate the detection and amplification of low-avidity T-cell specificities in biological samples using flow cytometry. KIH structures are described in the literature, e.g., in (6), (7), and U.S. Pat. App. Pub. 2018/0127481, “RECOMBINANT PMHC CLASS II MOLECULES,” each incorporated herein by reference in its entirety.

Production of soluble pMHCII molecules is more challenging than production of their pMHC class I counterparts because secreted MHCII α and β chains lacking the transmembrane and cytoplasmic domains do not form stable heterodimers, even in the presence of high affinity peptide ligands. The transmembrane regions of the MHCII α and β chains facilitate the proper assembly of the αβ heterodimer, presumably through the interaction of the two α-helical transmembrane segments (8). This challenge was addressed by replacing the transmembrane and cytoplasmic domains of MHCII chains by leucine zipper motifs (5). However, since MHCII-binding peptides play a critical role in the assembly and stabilization of the αβ heterodimer, this approach does not invariably support the expression of pMHCII monomers displaying epitopes with low affinity for MHC and/or the expression of MHCII types with peculiar structural features, such as certain HLA-DQ molecules (9). This represents a fundamental limitation for the use of these reagents as a tool to enumerate and track cognate autoreactive T-cells in autoimmunity, where many naturally-occurring autoimmune disease-relevant epitopes are weak MHCII binders. Another significant limitation of current pMHCII engineering approaches is that they are not suited for the production of pMHCII-based compounds at scale for therapeutic purposes. This is so because there are no orthogonal chromatographic separation schemes capable of purifying pMHCII complexes from eukaryotic cell culture supernatants with the degree of purity, yields and low costs required for clinical translation. Although for pure experimental purposes, this caveat can be addressed by addition of affinity separation tags into the pMHC complex, this practice is not acceptable for human translation as it bears the risk of triggering the generation of anti-drug antibodies.

Described herein is a novel pMHCII heterodimerization strategy that enables the production and purification, at high yields, of stable pMHCII monomers for a variety of applications. Also provided are pMHCII monomers wherein the transmembrane and cytoplasmic regions of the MHCII α and β chains are replaced by human or mouse IgG1-Fc modified to form knobs and holes, e.g., respectively. In some embodiments, these molecules are SDS stable, are expressed at significantly higher levels than conventional leucine-zippered pMHCIIs, and can be easily purified from culture supernatants using protein A/G chromatography without the need to include foreign, immunogenic affinity-purification tags in the molecule. In some embodiments, these molecules have superior TCR binding and triggering properties and, when used as multimeric structures to enumerate antigen-specific T-cells in complex biological samples, are amenable to signal amplification, including the use of anti-hFc antibodies. Collectively, the advantages of the molecular pMHCII engineering approach described herein can overcome the roadblocks that currently preclude the use of pMHCII designs for therapeutic applications. In some embodiments, the high expression yields of non peptide-tethered KIH-based pMHCs facilitates the screening of epitope libraries in the context of specific MHCII molecules and antigen receptors. Also provided are methods to generate stabilized difficult-to-express soluble TCRαβ heterodimers for multiple uses, including the identification of specific pMHC targets.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Materials and Methods

Mice. NOD/Lt mice were from the Jackson Lab (Bar Harbor, Me.). BDC2.5-NOD mice (expressing a transgenic T-cell receptor for the BDC2.5mi/IAg7 complex) are described in the literature, e.g., by (31).

pMHC production. Recombinant pMHCII were produced in CHO-S cells (Invitrogen) transduced with lentiviruses (Vector Builder, Chicago, Ill.) encoding a monocistronic message in which the peptide-MHCβ (or non-peptide-tethered MHCβ) and MHCα chains were separated by a ribosome skipping P2A-coding sequence, followed by an IRES-EGFP cassette (32). Alternatively, the peptide-MHCβ and MHCα chains were encoded in separate lentiviruses encoding IRES-EGFP and IRES-CFP cassettes, respectively. Peptide-tethered MHCII molecules were biotinylated in vitro, as described below. “Empty” MHCII molecules were biotinylated in vivo, by expressing the corresponding lentivirus-transduced constructs in BirA-transgenic CHO cells as described hereinbelow.

To express the various pMHCIIs, transduced CHO-S cells were grown in 2 L baffled flasks (Nalgene) in a shaker incubator at 125 rpm, 5% CO₂ and 37° C. Basal medium was Power-CHO-2 (Lonza) supplemented with 8 mM glutamine (Lonza) and gentamicin sulfate (0.05 mg/mL) (Lonza). The cultures were started in 400 mL of basal medium at 350,000-400,000 cells/mL and were supplemented with feeds: Cell Boost 7a (Hyclone) at 3% v/v and Cell Boost 7b (Hyclone) at 0.3% v/v on days 0, 3, 4, 5, 6, 8, 9 and 10. A temperature shift to 34° C. was done when cell densities reached 5-7×10⁶ cells/mL. Additional glutamine was added on day 7, to 2 mM. Glucose was added to 4.5 g/L when levels dropped below 3.5 g/L. Cells were harvested on Day 14 or when cell viability fell below 60%.

The secreted proteins were purified by sequential affinity chromatography on nickel and strep-tactin columns (for c-fos/c-jun-based pMHCII), protein A/G columns (for KIH-based pMHCII) and avidin columns (for in vivo-biotinylated empty KIH-based pMHCIIs after protein A/G purification) and used for NP coating or biotinylated in vitro (for peptide-tethered pMHCII) to produce pMHC tetramers using fluorochrome-conjugated streptavidin.

Molecular modelling. Molecular modelling was done with the DeepView-Swiss-PdbViewer software (33). KIH heterodimer modelling was based on the previously published crystal structure (34) (Protein Data Bank (PDB) ID: 4 NQS). The pMHCII cys-trap model was based on the previously published crystal structure of IA^{g7} complexed with GAD₂₀₇₋₂₂₀ (PDB ID: 1ES0) (35).

SDS-PAGE. The proteins were electrophoresed in 12% SDS-PAGE gels. To evaluate SDS stability of pMHCII monomers, samples were loaded with 0.83% SDS and were either boiled (100° C. for 5 minutes) or left unboiled. Fully denaturing conditions involved the addition of 20 mM (β-ME (Sigma).

In vitro biotinylation of pMHCII monomers. Biotinylation of pMHCII was done by using a biotin-protein ligase kit (BirA enzyme, Avidity). Briefly, 25 μM of pMHC was biotinylated with 10 μg of BirA enzyme in 50 mM bicine buffer pH 8.3 with 10 mM magnesium acetate, 10 mM ATP, and 85 μM of d-biotin at room temperature overnight. The reaction mixture was dialyzed against 20 mM Tris-HCl buffer pH8 and the resulting pMHCII was purified by ion exchange (mono-Q) chromatography. Biotin-conjugated pMHCII fractions were identified via ELISA using horseradish peroxidase-streptavidin (Sigma) and characterized via denaturing SDS-PAGE. The biotin-conjugated pMHCII fractions were pooled, buffer exchanged into PBS by spin ultrafiltration (Millipore, MW cut-off 30 KDa) and stored at −80° C.

pMHC tetramers. Phycoerythrin (PE)-conjugated tetramers were prepared using biotinylated pMHCII monomers and used to stain peripheral T-cells or TCR-transfected Jurkat cell lines as described (36, 37). “Empty” MHCII complexes were biotinylated in BirA-transgenic CHO-S cells and purified on avidin columns. Briefly, CHO-S cells were transduced with lentiviruses encoding each of the two chains of the KIH constructs as described above. BirA-ER enzyme (Addgene) was cloned into another lentiviral plasmid carrying human CD4 as a reporter gene and used to transduce CHO-S cell lines expressing the different KIH-based MHCIIs. Cells were FACS-sorted based on positivity for GFP, CFP and human CD4 using a Becton Dickinson FACsAria II sorter. Cell lines were expanded and grown to a density of 10-15⁷ cells/mL during 14 days in the presence of 2 μg/mL of biotin (ThermoFisher). Biotinylated soluble MHCII molecules were purified from culture supernatants by protein G column chromatography using an ÄKTA protein purification system (GE). PBS or 20 mM Trizma buffer exchange was done using a size exclusion column (GE). A second purification using an avidin column kit was done in order to purify in vivo biotinylated proteins (ThermoFisher).

The biotinylated molecules were then loaded with peptide by incubation with a 10-fold molar excess of PDC-E2₁₂₂₋₁₃₅ (Tebu-bio), PDC-E2₂₄₉₋₂₆₂ (Tebu-bio) and IGRP₁₃₋₂₅ (Genscript) in 100 mM NaPO4 pH6.0, 0.2% n-octyl-d-glucopyranoside (Sigma-Aldrich) and 1 mg/ml Pefabloc® (Sigma-Aldrich) for 72 hours at 37° C. The peptide-loaded MHCII molecules were then incubated with PE-streptavidin (ThermoFisher) at a 5:1 molar ratio overnight at room temperature to generate tetrameric pMHCII complexes.

Flow cytometry. To stain mononuclear cell suspensions from NOD mice, peripheral blood, splenocytes, lymph node and bone marrow cell suspensions were incubated with avidin for 15 min at room temperature and stained with tetramer (10-33 μg/mL, see below) in FACS buffer (0.05% sodium azide and 1% FBS in PBS) for 30 min at 4° C., washed, and incubated with FITC-conjugated anti-CD4 (5 μg/mL) and PerCP-conjugated anti-B220 (2 μg/mL; as a ‘dump’ channel) for 30 min at 4° C., in the presence of an anti-CD16/CD32 mAb (2.4G2; BD Pharmingen) to block FcRs. Cells were washed, fixed in 1% paraformaldehyde (PFA) in PBS and analyzed with FACScan, FACSaria, BD LSRII, FACSCanto or Fortessa flow cytometers. Analysis was done using FlowJo software.

TCR-transduced Jurkat cell lines were stained with 10 μg/ml of c-jun/c-fos-based pMHCII tetramer or 33 μg/ml KIH-based tetramer in 50 μl of PBS for 1 hour at 37° C. Propidium iodide (Sigma, St. Louis, Mo., USA) was added 5 min before analysis to discriminate live from dead cells.

For experiments using PBMCs spiked with clonal cells, experimental samples were created by mixing clonal T-cells (10⁴) with PBMCs (10⁶). The PBMCs were HLA-matched for the restricting HLA of the T-cell clone used. Some samples were treated prior to tetramer staining with the protein kinase inhibitor (PKI) Dasatinib (Axon Medchem) at 50 nM for 30 min at 37° C. Tetramer staining was performed with 33 μg/ml of peptide-loaded KIH-based pMHC tetramers in 50 μl of PBS for 1 hour at 37° C. All samples were subsequently stained with anti-CD4 (αCD4) (OKT4; BioLegend), αCD14 (HCD14; BioLegend) and, in some cases, with anti-human Fc-PE (Jackson ImmunoResearch) for 20 min at 37° C. Propidium iodide (Sigma) was added 5 min before analysis to discriminate live from dead cells.

Nanoparticle synthesis. Maleimide-functionalized, pegylated iron oxide NPs (PFM series) were produced in a single-step thermal decomposition in the absence of surfactants as described (38). Briefly, 3 g Maleimide-PEG (2 kDa MW, Jenkem Tech USA) were melted in a 50 mL round bottom flask at 100° C. and then mixed with 7 mL of benzyl ether and 2 mmol Fe(acac)₃. The reaction was stirred for 1 h and heated to 260° C. with reflux for 2 h. The mixture was cooled to room temperature and mixed with 30 mL water. Insoluble materials were removed by centrifugation at 2,000× g for 30 min. The NPs were purified using magnetic (MACS) columns (Miltenyi Biotec) and stored in water at room temperature or 4° C. The concentration of iron was determined spectrophotometrically at 410 nm in 2N hydrochloric acid (HCl).

pMHCII conjugation to NPs. pMHCII conjugation to maleimide-functionalized NPs (PF-M) was done via the free C-terminal Cys engineered into the MHCα chain/Knob. Briefly, pMHCs were mixed with NPs in 40 mM phosphate buffer, pH6.0, containing 2 mM ethylenediaminetetraacetic acid (EDTA), 150 mM NaCl, and incubated overnight at room temperature. pMHC-conjugated NPs were purified by magnetic separation and concentrated by ultrafiltration through Amicon Ultra-15 (100-300 kDa cut-off) and stored in PBS.

NP characterization. The size and dispersity of unconjugated and pMHCII-conjugated NPs were assessed via transmission electron microscopy (TEM, Hitachi H7650) and dynamic light scattering (DLS, Zetasizer, Malvern). Pegylated and pMHC-NPs were analyzed via 0.8% agarose gel electrophoresis, native and denaturing 10% SDS-PAGE. To quantify pMHC valency, we measured the pMHC concentration of the pMHC-NP preps using the Bradford assay (Thermo Scientific).

Reactivity of cys-trapped and non-cys-trapped KIH-based human pMHCIIs to conformation epitope-specific mAbs. The KIH-based pMHC monomers were diluted to an identical concentration (200 ng/mL) and serially diluted. A sandwich ELISA assay was used to capture and quantify the pMHCs. Briefly, plates were coated with goat anti-human IgG (Jackson ImmunoResearch) (working concentration 24 μg/mL) as a capture antibody. The capture antibody (100 μL/well) was incubated in a 96-well flat bottom Immuno plate (Thermo Scientific) overnight at room temperature. The plates were blocked using PBS containing 1% BSA and 0.05% sodium azide for 1 h. The plates were then washed 4 times with PBS containing 0.5% Triton X-100, 200 μL/well (washing buffer). The serially diluted pMHC-human KIH fusion protein solution (100 μL/well) was added to the wells and incubated for 2 h at room temperature. The plates were washed 4 times. The captured pMHCIIs were then detected using biotinylated anti-human HLA-DR mAb (clone L243, from Biolegend; 0.4 μg/well, 100 μL/well). The plates were incubated with the capture antibody for 2 h at room temperature, washed 4 times and then incubated with ExtrAvidin Peroxidase Conjugate (Sigma Aldrich; 1:2,000 dilution in PBS, 100 μL/well) for 30 min at room temperature. The plates were washed again, and incubated with 3, 3′, 5, 5′-Tetramethylbenzidine (TMB, Sigma-Aldrich; 100 μL/well) for 5 min. The color reaction was stopped by adding 50 μL of 2N H₂SO₄. The absorbance of the reaction was measured at 450 nm and 570 nm wavelengths using a plate reader (SpectraMax i3x, Molecular Devices).

TCR signaling in TCR/mCD4 or TCR/hCD4-transfected Jurkat cells. The TCRα and TCRβ cDNAs encoding the BDC2.5-TCR were generated from BDC2.5-CD4+ T-cell-derived mRNA using the 5′ RACE System for Rapid Amplification of cDNA Ends, version 2.0 kit (Thermo-Fisher Scientific), and subcloned as a P2A-tethered single open-reading frame into a retroviral vector upstream of an IRES-eGFP cassette. The TCR cDNAs encoding human IGRP₁₃₋₂₅/DR3-, PDC-E2₁₂₂₋₁₃₅/DRB4*0101/DRA1*-0101-, and PDC-E2₂₄₉₋₂₆₂/DRB4*0101/DRA1*-0101-specific TCRs were cloned from human T-cell clones generated from T1D or Primary Biliary Cholangitis patients as described (38). The human CD3+/TCRβ—JurMA (Jurkat) reporter cell line (expressing NFAT-driven luciferase) was transduced with retroviruses encoding murine or human CD4 and murine or human TCRαβ, respectively. eGFP and mouse or human CD4 double-positive cells were sorted by flow cytometry and stained with PE-labelled pMHCII tetramers to confirm specificity.

To measure NFAT-driven expression of luciferase, wild-type and BDC2.5/mCD4+ or IGRP₁₃₋₃₅/DR3-TCR/hCD4+ Jurkat cells were plated at 500,000 cells/mL in 200 μl of DMEM (Sigma-Aldrich) supplemented with 10% FBS (Sigma-Aldrich) in the presence or absence of 10 μg/mL of anti-hCD3ε mAb (OKT3, BD Biosciences) or various concentrations of pMHC-coated PFM for 12 h. Cells were washed 3 times with PBS and 10⁵ cells lysed in 20 μl Cell Culture Lysis Reagent (Promega) and incubated with 100 μl of Luciferase Assay Reagent (Promega) in opaque white plates (Greiner Bio One International GmbH) using a Veritas™ Microplate Luminometer (Promega) with injectors. Luciferase activity was expressed as relative luminescence units (RLUs), normalized to the luciferase activity of anti-CD3ε mAb-challenged cells.

pMHCII-NP therapy of NOD mice. Cohorts of 10-week-old female NOD mice were injected i.v. with pMHCII-coated NPs in PBS (20 μg pMHC/dose) twice a week for 5 weeks. Increases in the size of tetramer+ CD4+ T-cell pools in blood, spleen, lymph nodes and/or marrow, as well as their phenotypic properties, were assessed by flow cytometry as described (39).

Cytokine secretion assay. CD4+ T-cells from pMHC-NP-treated mice were enriched from spleen cell suspensions using a BD Imag enrichment kit, stained with pMHCII tetramers as described above and sorted into tetramer+ and tetramer− subsets by flow cytometry. FACS-sorted cells (2-3×10⁴) were stimulated with anti-CD3/anti-CD28 mAb-coated beads for 48 h and the supernatants collected 48 h later for measurement of cytokines via Luminex.

Statistical analyses. Quantitative data were compared by Mann-Whitney U or two-way ANOVA. Statistical significance was assumed at P<0.05.

### Example 1. pMHC Class II Molecule Constructs and Expression

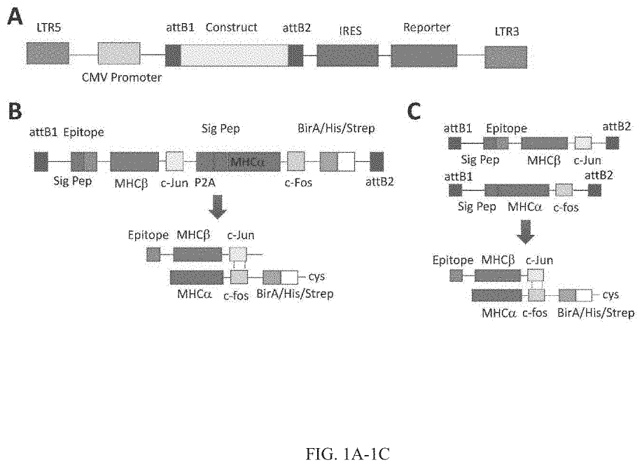

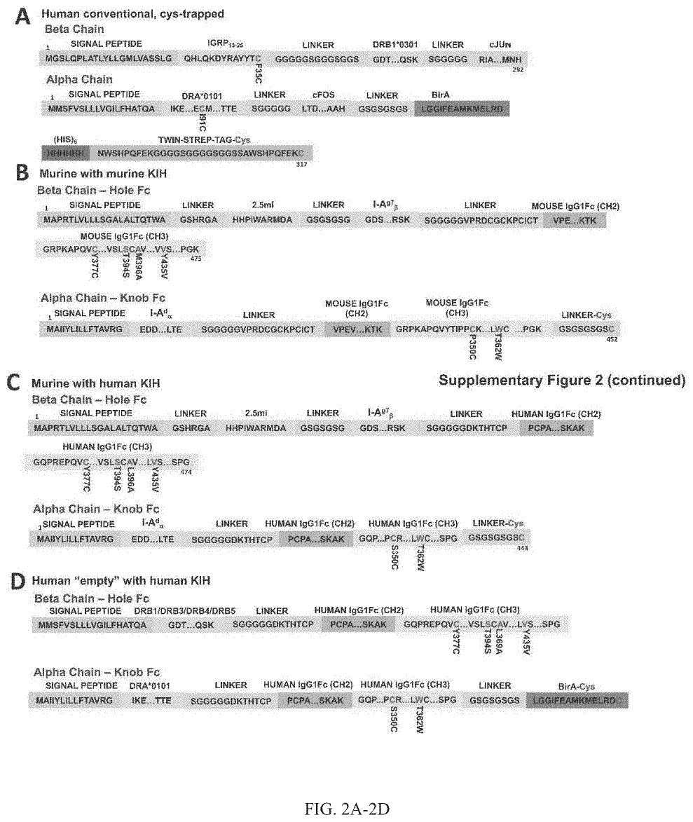

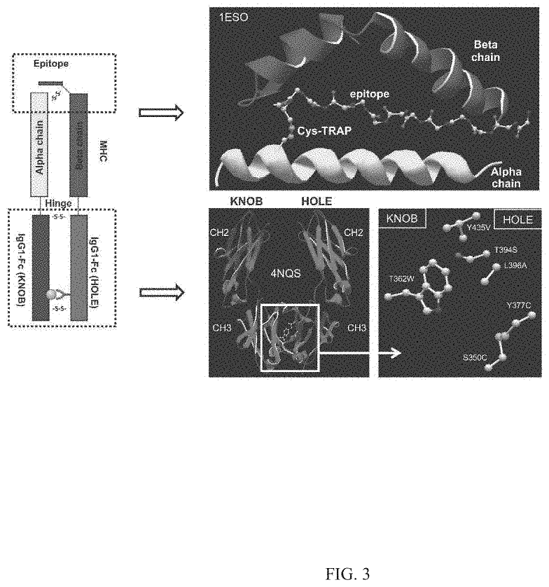

Lentiviral vectors encoding IRES-CFP or IRES-EGFP reporter cassettes (FIG. 1A) were used to express pMHCIIs in CHO cells. The pMHCIIα and β chains were either transcribed from a single ORF as two chains separated by a P2A ribosomal skipping sequence (FIG. 1B), or from two different ORFs in different vectors (FIG. 1C). FIGS. 2 and 3 summarize the structural features of representative constructs for the various pMHCIIs described here, as well as key junctional sequences. Table 1, generated using contemporary CHO cell cultures using representative cell lines, provides a list of the murine and human pMHCIIs used and their expression yields. Briefly, transduced CHO-S cells expressing high levels of EGFP and CFP were sorted by flow cytometry and grown in protein-free media in shake flasks using a fed batch protocol. pMHCIIs were purified from supernatants and used directly to coat iron oxide NPs, or were biotinylated to produce pMHCII tetramers.

TABLE 1
Peptides, MHC molecules, heterodimerization
domains and yields

| Tethered | | MHC | MHC | Hetero- | Yield |
|---|---|---|---|---|---|
| epitope | Sequence | beta | alpha | dimers | (mg/L) |
| BDC.2.5mi | HHPIWARMDA | I-Aβ^{g7} | I-Aα^{d} | JUN/FOS | 17.7 |
| | (SEQ ID | | | | |
| | NO: 1) | | | | |
| BDC.2.5mi | HHPIWARMDA | I-Aβ^{g7} | | HOLE/ | 80.6 |
| | (SEQ ID | | | KNOB | |
| | NO: 1) | | | | |
| TOPO | KLNYLDPRIT | I-Aβ^{b} | I-Aα^{d} | JUN/FOS | 2.3 |
| (722-736) | VAWCK | | | | |
| | (SEQ ID | | | | |
| | NO: 2) | | | | |
| ApoB | SQEYSGSVAN | I-Aβ^{b} | I-Aα^{b} | JUN/FOS | 0.38 |
| (3501- | EANVY | | | | |
| 3516) | (SEQ ID | | | | |
| | NO: 3) | | | | |
| mDSG3 | RNKAEFHQSV | I-Aβ^{b} | I-Aα^{b} | JUN/FOS | 0.2 |
| (301-315) | ISQYR | | | | |
| | (SEQ ID | | | | |
| | NO: 4) | | | | |
| IGRP | QHLQKDYRAY | DRB | DRA* | JUN/FOS | 12 |
| (13-25) | YTF | 1*0301 | 0101 | | |
| | (SEQ ID | | | | |
| | NO: 5) | | | | |
| IGRP | QHLQKDYRAY | DRB | DRA* | JUN/FOS | 4 |
| (13-25) | YTC | 1*0301 | 0101 | | |
| cystrap | (SEQ ID | | | | |
| | NO: 6) | | | | |
| IGRP | QHLQKDYRAY | DRB | DRA* | HOLE/ | 32.8 |
| (13-25) | YTF | 1*0301 | 0101 | KNOB | |
| | (SEQ ID | | | | |
| | NO: 5) | | | | |
| IGRP | QHLQKDYRAY | DRB | DRA* | HOLE/ | 95.8 |
| (13-25) | YTC | 1*0301 | 0101 | KNOB | |
| cystrap | (SEQ ID | | | | |
| | NO: 6) | | | | |
| PPI | SLQPLALEGS | DRB | DRA* | JUN/FOS | 4.12 |
| (76-90) | LQSRC | 1*0401 | 0101 | | |
| 88S | (SEQ ID | | | | |
| cystrap | NO: 7) | | | | |
| PPI | SLQPLALEGS | DRB | DRA* | HOLE/ | 44.5 |
| (76-90) | LQSRG | 1*0401 | 0101 | KNOB | |
| 88S | (SEQ ID | | | | |
| | NO: 8) | | | | |
| IGRP | YTFLNFMSNV | DRB | DRA* | JUN/FOS | 0.6 |
| (23-35) | GDP | 1*0401 | 0101 | | |
| | (SEQ ID | | | | |
| | NO: 9) | | | | |
| IGRP | YTFLNFMSNV | DRB | DRA* | JUN/FOS | 45.9 |
| (23-35) | GDC | 1*0401 | 0101 | | |
| cystrap | (SEQ ID | | | | |
| | NO: 10) | | | | |
| Glia | QPFPQPELPY | DQB | DQA1* | HOLE/ | |
| (57-68) | GC | 1*0201 | 0501 | KNOB | |
| Cystrap | (SEQ ID | | | | |
| | NO: 11) | | | | |
| Glia | PQPELPYPQ | DQB | DQA1* | JUN/FOS | 2.6 |
| (62-72) | PC | 1*0201 | 0501 | | |
| cystrap | (SEQ ID | | | | |
| | NO: 12) | | | | |
| Glia | PQPELPYPQ | DQB | DQA1* | HOLE/ | 30.4 |
| (62-72) | PE | 1*0201 | 0501 | KNOB | |
| | (SEQ ID | | | | |
| | NO: 13) | | | | |
| Glia | PQPELPYPQ | DQB | DQA1* | HOLE/ | 39.5 |
| (62-72) | PC | 1*0201 | 0501 | KNOB | |
| cystrap | (SEQ ID | | | | |
| | NO: 12) | | | | |
| NONE | — | DRB | DRA* | HOLE/ | 10.2 |
| | | 1*0301 | 0101 | KNOB | |
| NONE | — | DRB | DRA* | HOLE/ | 29.3 |
| | | 4*0101 | 0101 | KNOB | |
| NONE | — | DRB | DRA* | HOLE/ | 29.4 |
| | | 5*0101 | 0101 | KNOB | |
| NONE | — | DRB | DRA* | HOLE/ | 22.9 |
| | | 1*1501 | 0101 | KNOB | |

### Example 2. Production of Cys-Trapped pMHC Class II Molecules

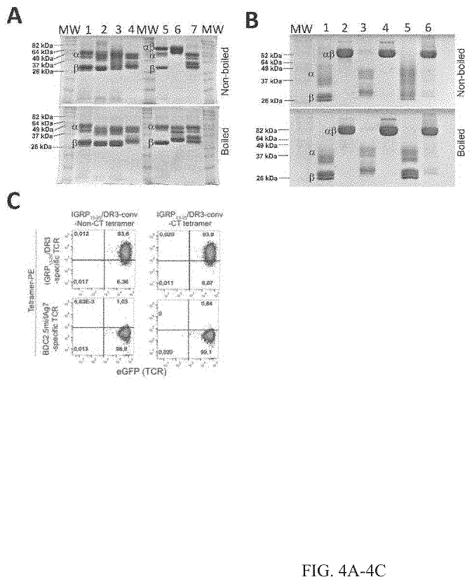

It has been shown that the epitope is a major stabilizer of soluble pMHCII heterodimers (10, 11). Peptides binding with high affinity support higher heterodimer stability than those binding with low affinity. However, intrinsic molecular properties of allelic MHCII molecules also play a major role in defining the stability of pMHCIIs, independently of the peptide (12, 13). As a result, whereas certain pMHCII molecules migrate as a single, large molecular species in non-denaturing SDS-PAGE, most others melt into single α and β chains (FIG. 4A) and are expressed at low yields (Table 1) or not at all (not shown). We thus reasoned that we could increase the stability and possibly the production yields of SDS-unstable c-jun/c-fos-zippered MHCIIs (herein also referred to ‘conventional’) by introducing cysteines at appropriate positions in the peptide and the MHCIIα chain to anchor the peptide onto the MHC on a preferred binding register (14, 15) (herein referred to as cys-trapping (CT)). We note that our prior attempts to address this issue by introducing artificial disulphide bonds at or near the c-jun/c-fos zipper in poorly-expressing pMHCII constructs were unsuccessful (not shown). We first focused on the type 1 diabetes (T1D)-relevant IGRP₁₃₋₂₅/DRB1*0301/DRA1*0101 complex (Table 1). We replaced a C-terminal phenylalanine in IGRP₁₃₋₂₅ and a proximal serine in the MHCIIα chain for cysteines (FIGS. 2A and 3). This resulted in SDS stability (FIG. 4B) without any appreciable loss of cognate T-cell binding efficiency, as measured using pMHC tetramers and a human CD4/TCR-transduced Jurkat cell line (FIG. 4C). Similar results were obtained with other pHLA molecules, such as IGRP₂₃₋₃₅/DRB1*0401/DRA1*0101 (FIG. 4B). The use of a cys-trap also enabled the production of much more difficult-to-express HLA molecules, such as HLA-DQB1*0201/DQA1*0501 displaying gliadin residues 62-72 (Table 1 and FIG. 4B). Cys-trapping, however, increased production yields for some but not all pMHCs (e.g. IGRP₁₃₋₂₅/DRB1*0301/DRA1*0101) (Table 1). Furthermore, cys-trapping cannot be adopted by all pMHCIIs, because introduction of artificial cysteines within the peptide might in some cases impair T-cell binding and/or activation, and because epitopes that already contain naturally-occurring cysteines within their sequence are not suitable for this approach.

### Example 3. Production of Knob in Hole pMHC Class II Molecules

To address this and other limitations of current pMHCII production strategies, including heterodimer instability, discrete production yields, and the lack of efficient and scalable purification schemes broadly applicable to any pMHC type (for human in vivo use), we explored the feasibility of using a knob-into-hole (KIH)-IgG-based heterodimerization strategy. Introduction of complementary amino acid substitutions in the CH3 domain of the Fc region of human IgG1 (or other IgG subtypes) results in the generation of two different Fc molecules (knob and hole) with favourable heterodimerization and unfavourable homodimerization potential (6, 7). We reasoned that, unlike Fc-fusion-based pMHC dimerization, which generates large Ig-like molecular structures in which αβ heterodimer formation and stability still require the use of leucine zippers and are regulated by the same principles that control the assembly of non-Fc-fused, c-jun/c-fos zippered pMHCIIs (16-19), KIH-based pMHCII heterodimerization would potentially render pMHCIIs intrinsically more stable with only a relatively minor increase in total molecular weight.

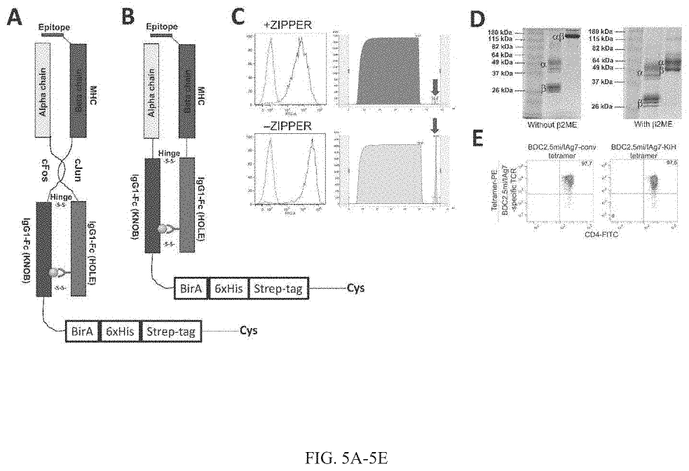

We tethered the murine IAα^{d} chain with a modified Fc region of human IgG1 to behave as a knob (both with and without the c-fos motif), and the corresponding IAβ^{g7} chain (with and without the c-jun motif) to the Fc region of human IgG1 modified to behave as a hole (FIGS. 5A-5B and FIG. 2C). In our initial designs, we also included a BirA biotinylation site, a 6× histidine and twin strep tags, and a cysteine at the C-terminal end of the knob, generating a ‘knob’ that is larger than its ‘hole’ counterpart. Both the leucine-zippered and non-zippered cell lines expressed the transgenic RNA, as documented by the expression of EGFP (FIG. 5C, left), but only the latter secreted protein G-binding material in the supernatant (FIG. 5C, right), which ran as a single band in native SDS-PAGE (FIG. 5D left panel), and as two separate bands of different molecular weight, as expected, but similar intensity in denaturing SDS-PAGE (FIG. 5D, right panel), suggesting ˜1:1 stoichiometries. The KIH version of this pMHCII expressed at >4-fold higher levels than its non-KIH-based counterpart (Table 1). These molecules folded appropriately because pMHCII tetramers generated with these KIH-based pMHCII monomers stained splenic CD4+ T-cells from a transgenic mouse expressing a BDC2.5mi-specific T-cell receptor (TCR) essentially like its zippered, non-KIH-based counterpart (FIG. 5E).

### Example 4. Effect of KIH pMHC Class II Nanoparticles on TCR Signaling

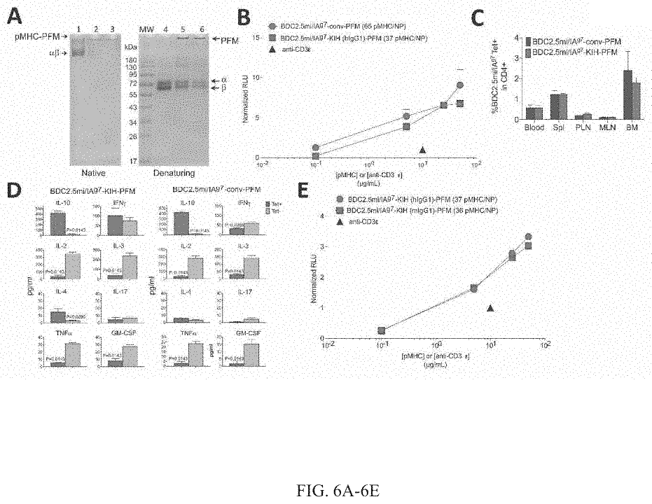

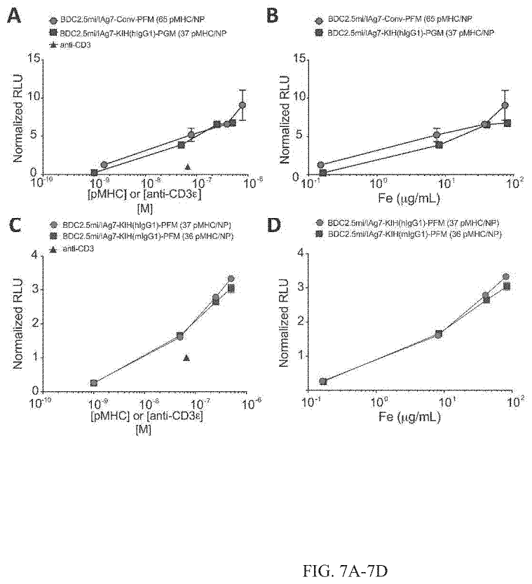

When delivered systemically, NPs coated with autoimmune disease relevant pMHCII (pMHC-NP) can re-program (and expand) autoantigen experienced effector/memory T-cells into cognate T-regulatory type 1 (TR1) cells, leading to reversal of various autoimmune diseases (2, 4). The biological potency of these compounds (TR1 cell formation in vivo) is a function of pMHC valency on the NP surface and can be gauged in vitro using reporter cell lines (3). We thus compared the TCR signaling potency of NPs coated with non-KIH-based BDC2.5mi/IA^{g7} pMHC (at 65 pMHCs/NP) with NPs coated with its KIH-based counterpart (at 37 pMHCs/NP) (FIG. 6A), on Jurkat cells co-expressing mouse CD4, a cognate TCR and NFAT-driven luciferase. Both compounds had similar potency, despite carrying significantly different pMHC valencies (FIG. 6B and FIGS. 7A-7B).

Similar results were obtained in vivo; the KIH-based pMHCII-NP compounds triggered the formation and expansion of similar numbers of cognate (tetramer+) TR1 cells as their non-KIH-based counterparts (FIGS. 6C and 6D). Similar results were obtained when we produced BDC2.5-I-Aβ^{g7}-Hole/I-Aα^{d}-Knob heterodimers using murine rather than human IgG1-Fc-based knobs and holes (FIG. 2E, FIGS. 2B and 7C-7D).

### Example 5. Stabilization of Weak Interactions

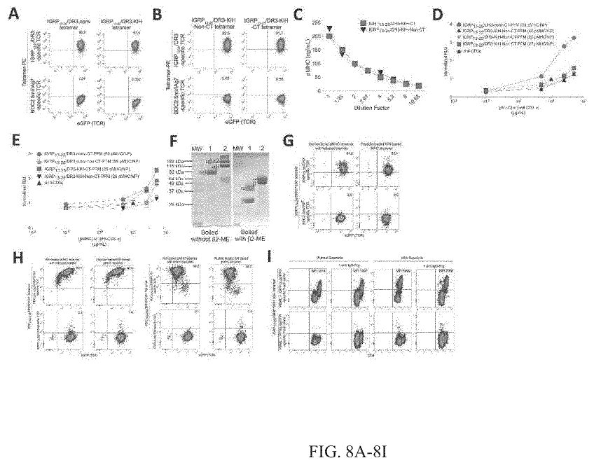

We next asked if this KIH strategy could also be used to stabilize weaker peptide:MHC interactions, such as IGRP₁₃₋₂₅/DRB1*0301/DRA1*0101. As was the case for zippered BDC2.5-I-Aβ^{g7}-Hole/I-Aα^{d}-Knob heterodimers, zippered IGRP₁₃₋₂₅-DRB1*0301-Hole/DRA1*0101-Knob heterodimers could not be expressed, but removal of the c-jun/c-fos zipper from the molecule led to efficient expression, at levels significantly greater than those obtained from CHO-S cells secreting non-KIH-based IGRP₁₃₋₂₅-DRB1*0301/DRA1*0101 heterodimers (Table 1). pMHCII tetramers produced with the KIH-based monomers stained cognate T-cells essentially like tetramers produced using leucine-zippered pMHCII monomers (FIG. 8A). Addition of the cys-trap register fixing mutations in the peptide and MHCIIα chain of these complexes (FIGS. 2A and 3) further increased expression yields (Table 1). This molecular modification did not disrupt the TCR-binding properties of these molecules because staining of cognate TCR-expressing Jurkat cells with tetramers made with the CT vs non-CT KIH-based constructs was essentially equivalent (FIG. 8B). Furthermore, these molecules reacted quantitatively equally to an anti-DR mAb (clone L243) that binds to a conformational epitope on the HLA-DRα chain that requires the correct folding of the αβ heterodimer (20, 21) (FIG. 8C).

### Example 6. Increased TCR Signaling Potency

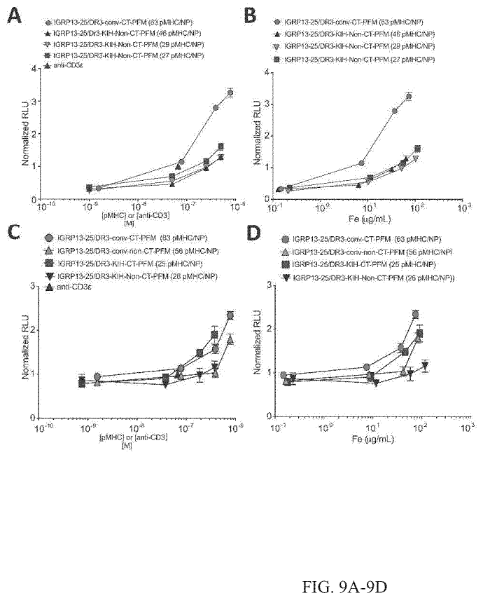

The above data suggested that introduction of a cys-trap between IGRP₁₃₋₂₅ and DR3 increases the structural stability of the heterodimer and pMHC production yields without interfering with TCR binding. However, when we compared the in vitro potency of NPs coated with a cys-trapped version of the non-KIH-based human IGRP₁₃₋₂₅/DRB1*0301-DRA*0101 pMHC (at 63 pMHCs/NP) with that of NPs coated with three non-cys-trapped KIH-based IGRP₁₃₋₂₅/DRB1*0301-DRA*0101 preparations (at 46, 29 and 27 pMHCs/NP), the latter three elicited significantly reduced luciferase responses from cognate Jurkat cells (FIG. 8D and FIGS. 9A-9B). The following three lines of evidence suggested the possibility that these differences might be accounted for by the presence of the cys-trap in the non-KIH-based pMHC that was used as a control. First, NP preparations displaying low valencies of the KIH-based BDC2.5mi/IA^{g7} pMHC performed essentially like NPs displaying high valencies of its zippered, non-KIH-based counterpart (FIG. 6B and FIGS. 7A-7B), suggesting that KIH-based pMHCs support increased TCR signaling. Second, all three NP preparations displaying the non-cys-trapped KIH-based IGRP₁₃₋₂₅/DRB1*0301-DRA*0101 also performed similarly in this assay, in a valency-independent manner (from 27-46 pMHCs/NP), consistent with the hypothetical increased potency of KIH-based designs. To investigate this hypothesis, we compared the biological potency of NPs coated with cys-trapped and non-cys-trapped versions of both types of pMHC constructs (non-KIH-based, and KIH-based). Surprisingly, with both construct types, inclusion of a cys-trap boosted potency (FIG. 8E and FIGS. 9C-9D). Indeed, NPs coated with the cys-trapped KIH-based construct had similar function as NPs coated with the cys-trapped non-KIH-based construct, despite significant differences in pMHC valency (56 and 63 for cys-trapped and non-cys-trapped non-KIH-based pMHC, respectively, vs. 25 and 26 for cys-trapped and non-cys-trapped KIH-based pMHC, respectively), again supporting the idea that the use of KIH-based pMHCs on NPs lowers the pMHC valency threshold required for biological activity.

### Example 7. Production of Peptide-HLA-DQ Molecules

Peptide-HLA-DQ complexes are difficult to express (9). As noted above, we could only produce significant amounts of c-jun/c-fos-zippered Gliadin₆₂₋₇₂/DQB1*0201/DQA1*0501 when the peptide was cys-trapped onto the MHC molecule, albeit at low yields (Table 1 and FIG. 4B). Remarkably, substitution of the leucine zipper domain with a KIH enabled the production of Gliadin₆₂₋₇₂/DQB1*0201/DQA1*0501 by CHO-S cells at yields that were 15-fold higher (Table 1 and FIG. 8F).

### Example 8. Production and Antigen Loading of “Empty” pMHC Class II Molecules

Some experimental approaches for T-cell epitope mapping require the use of extensive arrays of pMHCII tetramers to identify epitope reactivity by flow cytometry (22-24). In this context, the use of pMHCII molecules displaying covalently tethered peptides is not practical, as it implies purifying many different pMHCII molecules and generating the corresponding fluorochrome-labeled tetramers for each specific epitope. We thus investigated if the KIH-based approach could also be used to express high levels of non-peptide-tethered pMHCIIs from CHO cells and whether these compounds could be used for peptide-loading in vitro (25, 26). As shown in Table 1, transduced CHO-S cells secreted high levels of 4 different non-peptide-tethered human DRB types, including DRB1*0301/DRA1*0101, DRB4*0101/DRA*0101, DRBS*0101/DRA*0101 and DRB1*1501/DRA*0101. Importantly, these complexes could be loaded with peptides in vitro and the corresponding tetramers bound to cognate T-cells essentially like their peptide-tethered counterparts (FIGS. 8G and 8H).

Different strategies have been described to increase the staining intensity cognate T-cells with fluorochrome-labelled pMHC multimers (27), including the use of kinase inhibitors, the formation of cooperative pMHC/TCR clusters with crosslinking antibodies (28), and the use of scaffolds enabling the production of higher-order multimeric structures such as dextramers (29). This is particularly useful in autoimmune diseases, where the peripheral frequencies of autoreactive T-cells and their avidity for cognate pMHC complexes are significantly lower than those seen for foreign antigen-specific T-cells, such as in the context of infection and allergy. We thus investigated whether the signal-to-noise ratio of cognate T-cell staining with pMHCII tetramers could be improved using anti-hIgG-based amplification of KIH-based pMHCII tetramer binding. Human PBMCs were spiked with human clonal IGRP13-25/DR3-specific CD4+ T-cells and stained with cognate KIH-based pMHCII tetramers in the presence or absence of the protein kinase inhibitor Dasatinib (to inhibit TCR downregulation) followed by anti-hIgG-PE amplification. As shown in FIG. 8I, anti-hIgG increased the mean fluorescence signal intensity of tetramer staining, both in the presence and absence of Dasatinib.

### REFERENCES

1. Altman, J. D. et al. Phenotypic analysis of antigen-specific T lymphocytes. Science 274, 94-96 (1996).

2. Clemente-Casares, X. et al. Expanding antigen-specific regulatory networks to treat autoimmunity. Nature 530, 434-440 (2016)

3. Singha, S. et al. Peptide-MHC-based nanomedicines for autoimmunity function as T-cell receptor microclustering devices. Nat. Nanotech. 12, 701-710 (2017).

4. Umeshappa, C. S. et al. Supression of a broad spectrum of liver autoimmune pathologies by single peptide-MHC-based nanomedicines. Nature Comm 10, 1-17. (2019)

5. Vollers, S. S. & Stern, L. J. Class II major histocompatibility complex tetramer staining: progress, problems, and prospects. Immunology 123, 305-313 (2008).

6. Merchant, A. M. et al. An efficient route to human bispecific IgG. Nat. Biotechnol. 16, 677-681 (1998).

7. Carter, P. Bispecific human IgG by design. J. Immunol. Methods 248, 7-15 (2001).

8. Cosson, P. & Bonifacino, J. S. Role of transmembrane domain interactions in the assembly of class II MHC molecules. Science 258, 659-662 (1992).

9. Quarsten, H. et al. Staining of celiac disease-relevant T cells by peptide-DQ2 multimers. J. Immunol. 167, 4861-4868 (2001).

10. Sadegh-Nasseri, S. & Germain, R. N. A role for peptide in determining MHC class II structure. Nature 353, 167-170 (1991).

12. Sadegh-Nasseri, S. & Germain, R. N. How MHC class II molecules work: peptide-dependent completion of protein folding. Immunol. Today 13, 43-46 (1992).

12. Verreck, F. A. et al. The generation of SDS-stable HLA DR dimers is independent of efficient peptide binding. Int. Immunol. 8, 397-404 (1996).

13. Nelson, C. A., Petzold, S. J. & Unanue, E. R. Identification of two distinct properties of class II major histocompatibility complex-associated peptides. Proc. Natl. Acad. Sci. U.S.A. 90, 1227-1231 (1993).

14. Stadinski, B. D. et al. Diabetogenic T cells recognize insulin bound to IAg7 in an unexpected, weakly binding register. Proc. Natl. Acad. Sci. U.S.A. 107, 10978-10983 (2010).

15. Yang, J. et al. Autoreactive T cells specific for insulin B:11-23 recognize a low-affinity peptide register in human subjects with autoimmune diabetes. Proc. Natl. Acad. Sci. U.S.A. 111, 14840-14845 (2014).

16. Casares, S. et al. Down-regulation of diabetogenic CD4+ T cells by a soluble dimeric peptide-MHC class II chimera. Nat. Immunol. 3, 383-391 (2002).

17. Preda, I. et al. Soluble, dimeric HLA DR4-peptide chimeras: an approach for detection and immunoregulation of human type-1 diabetes. Eur. J. Immunol. 35, 2762-2775 (2005).

18. Moro, M. et al. Generation of functional HLA-DR*1101 tetramers receptive for loading with pathogen- or tumour-derived synthetic peptides. BMC Immunol. 6, 24 (2005).

19. Goldberg, B. & Bona, C. Dimeric MHC-peptides inserted into an immunoglobulin scaffold as new immunotherapeutic agents. J. Cell. Mol. Med. 15, 1822-1832 (2011).

20. Gorga, J. C., Horejsi, V., Johnson, D. R., Raghupathy, R. & Strominger, J. L. Purification and characterization of class II histocompatibility antigens from a homozygous human B cell line. J. Biol. Chem. 262, 16087-16094 (1987).

21. Stockel, J. et al. Refolding of human class II major histocompatibility complex molecules isolated from Escherichia coli. Assembly of peptide-free heterodimers and increased refolding-yield in the presence of antigenic peptide. J. Biol. Chem. 269, 29571-29578 (1994).

22. James, E. A. et al. Tetramer-guided epitope mapping reveals broad, individualized repertoires of tetanus toxin-specific CD4+ T cells and suggests HLA-based differences in epitope recognition. Int. Immunol. 19, 1291-1301 (2007).

23. Novak, E. J. et al. Tetramer-guided epitope mapping: rapid identification and characterization of immunodominant CD4+ T cell epitopes from complex antigens. J. Immunol. 166, 6665-6670 (2001).

24. Novak, E. J., Liu, A. W., Nepom, G. T. & Kwok, W. W. MHC class II tetramers identify peptide-specific human CD4(+) T cells proliferating in response to influenza A antigen. J. Clin. Invest. 104, R63-67 (1999).

25. Wallny, H. J., Sollami, G. & Karjalainen, K. Soluble mouse major histocompatibility complex class II molecules produced in Drosophila cells. Eur. J. Immunol. 25, 1262-1266 (1995).

26. Yang, J., Jaramillo, A., Shi, R., Kwok, W. W. & Mohanakumar, T. In vivo biotinylation of the major histocompatibility complex (MHC) class II/peptide complex by coexpression of BirA enzyme for the generation of MHC class II/tetramers. Hum. Immunol. 65, 692-699 (2004).

27. Dolton, G. et al. More tricks with tetramers: a practical guide to staining T cells with peptide-MHC multimers. Immunology 146, 11-22 (2015).

28. Lissina, A. et al. Protein kinase inhibitors substantially improve the physical detection of T-cells with peptide-MHC tetramers. J. Immunol. Methods 340, 11-24 (2009).

29. Batard, P. et al. Dextramers: new generation of fluorescent MHC class I/peptide multimers for visualization of antigen-specific CD8+ T cells. J. Immunol. Methods 310, 136-148 (2006).

30. Crawford, F. et al. Use of baculovirus MHC/peptide display libraries to characterize T-cell receptor ligands. Immunol. Rev. 210, 156-170 (2006).

31. Katz, J. D., Wang, B., Haskins, K., Benoist, C. & Mathis, D. Following a diabetogenic T cell from genesis through pathogenesis. Cell 74, 1089-1100 (1993).

32. Hoist, J. et al. Generation of T-cell receptor retrogenic mice. Nat. Protoc. 1, 406-417 (2006).

33. Guex, N. & Peitsch, M. C. SWISS-MODEL and the Swiss-PdbViewer: an environment for comparative protein modeling. Electrophoresis 18, 2714-2723 (1997).

34. Elliott, J. M. et al. Antiparallel conformation of knob and hole aglycosylated half-antibody homodimers is mediated by a CH2-CH3 hydrophobic interaction. J. Mol. Biol. 426, 1947-1957 (2014).

35. Corper, A. L. et al. A structural framework for deciphering the link between I-Ag7 and autoimmune diabetes. Science 288, 505-511 (2000).

36. Amrani, A. et al. Progression of autoimmune diabetes driven by avidity maturation of a T-cell population. Nature 406, 739-742 (2000).

37. Stratmann, T. et al. The I-Ag7 MHC class II molecule linked to murine diabetes is a promiscuous peptide binder. J. Immunol. 165, 3214-3225 (2000).

38. Singha, S. et al. Peptide-MHC-based nanomedicines for autoimmunity function as T-cell receptor microclustering devices. Nat. Nanotech. 12, 701-710 (2017).

39. Clemente-Casares, X. et al. Expanding antigen-specific regulatory networks to treat autoimmunity. Nature 530, 434-440 (2016).

Each incorporated herein by reference in its entirety.

1. An isolated pMHC monomer, wherein the pMHC monomer is a pMHC class II monomer comprising a first polypeptide and a second polypeptide, wherein:
the first polypeptide and the second polypeptide meet at an interface, wherein the interface of the first polypeptide comprises an engineered protuberance which is positionable in an engineered cavity in the interface of the second polypeptide;
and wherein
(i) the first polypeptide comprises an MHC class II α1 domain, an MHC class II α2 domain, or a combination thereof, a first antibody C_{H}2 domain, and a first antibody C_{H}3 domain; and the second polypeptide comprises an MHC class II β1 domain, an MHC class II β2 domain, or a combination thereof, a second antibody C_{H}2 domain, and a second antibody C_{H}3 domain;
wherein a disease-relevant antigen is connected to the MHC class II α1 domain or the MHC class II β1 domain by a flexible linker;
or
(ii) the first polypeptide comprises an MHC class II β1 domain, an MHC class II β2 domain, or a combination thereof, a first antibody C_{H}2 domain, and a first antibody C_{H}3 domain, and the second polypeptide comprises an MHC class II α1 domain, an MHC class II α2 domain, or a combination thereof, a second antibody C_{H}2 domain, and a second antibody C_{H}3 domain;
wherein a disease-relevant antigen is connected to the MHC class II α1 domain or the MHC class II β1 domain by a flexible linker;
wherein in (i) or (ii) the engineered protuberance of the first polypeptide is in the first C_{H}3 domain, and the engineered cavity of the second polypeptide is in the second C_{H}3 domain.

2. An isolated pMHC monomer, wherein the pMHC monomer is a pMHC class I monomer comprising a first polypeptide and a second polypeptide, wherein:
the first polypeptide and the second polypeptide meet at an interface, wherein the interface of the first polypeptide comprises an engineered protuberance which is positionable in an engineered cavity in the interface of the second polypeptide;
and wherein
(i) the first polypeptide comprises a β2-microglobulin domain, an MHC class I α1 domain, an MHC class I α2 domain, an MHC class I α3 domain, a first antibody C_{H}2 domain, and a first antibody C_{H}3 domain, and the second polypeptide comprises a second antibody C_{H}2 domain, and a second antibody C_{H}3 domain;
wherein a disease-relevant antigen is connected to the β2-microglobulin domain by a flexible linker;
or
(ii) the first polypeptide comprises a first antibody C_{H}2 domain, and a first antibody C_{H}3 domain, and the second polypeptide comprises a β2-microglobulin domain, an MHC class I α1 domain, an MHC class I α2 domain, an MHC class I α3 domain, a second antibody C_{H}2 domain, and a second antibody C_{H}3 domain;
wherein a disease-relevant antigen is connected to the β2-microglobulin domain by a flexible linker;
wherein in (i) or (ii) the engineered protuberance of the first polypeptide is in the first C_{H}3 domain, and the engineered cavity of the second polypeptide is in the second C_{H}3 domain.

3-12. (canceled)

13. A polynucleotide encoding the first or the second polypeptide of claim 1.

14. A host cell comprising the polynucleotide of claim 13.

15-25. (canceled)

26. A method for production and purification of the isolated pMHC monomer of claim 1, comprising the steps of:
a) culturing a host cell comprising a nucleic acid encoding the first and second polypeptide; and
b) purifying the pMHC monomer from the host cell culture;
or the steps of:
a) culturing a first host cell comprising a nucleic acid encoding the first polypeptide;
b) culturing a second host cell comprising a nucleic acid encoding the second polypeptide;
c) purifying the polypeptides from the first and second host cell cultures;
and
d) forming the purified pMHC monomer by incubating the first and second polypeptides together.

27-32. (canceled)

33. A high potency receptor-signaling pMHC monomer-nanoparticle conjugate, comprising a nanoparticle core coupled to a plurality of isolated pMHC monomers of claim 1, optionally wherein the pMHC monomers are coupled to the nanoparticle at a low valency or low density, and wherein the plurality of pMHC monomers comprises one or more pMHC monomer species, wherein each pMHC monomer species comprises a different disease-relevant antigen.

34-39. (canceled)

40. A method for making a high potency receptor-signaling pMHC monomer-nanoparticle conjugate comprising: coupling a nanoparticle core to a plurality of isolated pMHC monomers of claim 1, optionally wherein the pMHC monomers are coupled to the nanoparticle at a low valency or low density, and wherein the plurality of pMHC monomers comprises one or more pMHC monomer species, wherein each pMHC monomer species comprises a different disease-relevant antigen.

41-58. (canceled)

59. A method for high-yield production and purification of an MHC monomer, wherein the MHC monomer is an MHC class II monomer comprising a first polypeptide and a second polypeptide, wherein:
the first polypeptide and the second polypeptide meet at an interface, wherein the interface of the first polypeptide comprises an engineered protuberance which is positionable in an engineered cavity in the interface of the second polypeptide;
and
(i) the first polypeptide comprises an MHC class II α1 domain, an MHC class II α2 domain, or a combination thereof, a first antibody C_{H}2 domain, and a first antibody C_{H}3 domain; and the second polypeptide comprises an MHC class II β1 domain, an MHC class II β2 domain, or a combination thereof, a second antibody C_{H}2 domain, and a second antibody C_{H}3 domain; or
(ii) the first polypeptide comprises an MHC class II β1 domain, an MHC class II β2 domain, or a combination thereof, a first antibody C_{H}2 domain, and a first antibody C_{H}3 domain; and the second polypeptide comprises an MHC class II al domain, an MHC class II α2 domain, or a combination thereof, a second antibody C_{H}2 domain, and a second antibody C_{H}3 domain;
wherein in (i) or (ii) the engineered protuberance of the first polypeptide is in the first C_{H}3 domain, and the engineered cavity of the second polypeptide is in the second C_{H}3 domain,
the method comprising the steps of:
a) culturing a host cell comprising a nucleic acid encoding the first and second polypeptide; and
b) purifying the MHC class II monomer from the host cell culture;
or the steps of:
a) culturing a first host cell comprising a nucleic acid encoding the first polypeptide;
b) culturing a second host cell comprising a nucleic acid encoding the second polypeptide;
c) purifying the polypeptides from the first and second host cell cultures;
and
d) forming the MHC class II monomer by incubating the first and second polypeptides together.

60. A method for high-yield production and purification of an MHC monomer, wherein the MHC monomer is an MHC class I monomer comprising a first polypeptide and a second polypeptide, wherein:
the first polypeptide and the second polypeptide meet at an interface, wherein the interface of the first polypeptide comprises an engineered protuberance which is positionable in an engineered cavity in the interface of the second polypeptide;
and
(i) the first polypeptide comprises an MHC class I α2 domain, an MHC class I α3 domain, or a combination thereof, a first antibody C_{H}2 domain, and a first antibody C_{H}3 domain; and the second polypeptide comprises an MHC class I α1 domain, a β-microglobulin, or a combination thereof, a second antibody C_{H}2 domain, and a second antibody C_{H}3 domain; or
(ii) the first polypeptide comprises an MHC class I α1 domain, a β-microglobulin domain, or a combination thereof, a first antibody C_{H}2 domain, and a first antibody C_{H}3 domain; and the second polypeptide comprises an MHC class I α2 domain, an MHC class I α3 domain, or a combination thereof, a second antibody C_{H}2 domain, and a second antibody C_{H}3 domain;
wherein in (i) or (ii) the engineered protuberance of the first polypeptide is in the first C_{H}3 domain, and the engineered cavity of the second polypeptide is in the second C_{H}3 domain,
the method comprising the steps of:
a) culturing a host cell comprising a nucleic acid encoding the first and second polypeptide; and
b) purifying the MHC class I monomer from the host cell culture;
or the steps of:
a) culturing a first host cell comprising a nucleic acid encoding the first polypeptide;
b) culturing a second host cell comprising a nucleic acid encoding the second polypeptide;
c) purifying the polypeptides from the first and second host cell cultures; and
d) forming the MHC class I monomer by incubating the first and second polypeptides together.

61-69. (canceled)

70. An MHC monomer produced using the method of claim 59.

71. An MHC monomer produced using the method of claim 60.

72. A pMHC class I monomer or MHC class I monomer of claim 2, wherein the MHC comprises all or part of a HLA-A, HLA-B, HLA-C, HLA-E, HLA-F, HLA-G, or CD1-like (non-classical) molecule.

73. A pMHC class II monomer or MHC class II monomer of claim 1, wherein the MHC comprises all or part of a HLA-DR, HLA-DQ, HLA-DP, HLA-DM, HLA-DOA, or HLA-DOB molecule.

74. A method for making a pMHC class I or class II multimer, the method comprising multimerizing a plurality of pMHC class I or class II monomers of claim 1.

75. A pMHC multimer comprising a pMHC monomer of claim 1.

76. A pMHC multimer made by the method of claim 74.

77-81. (canceled)

82. A method of making an MHC multimer, the method comprising multimerizing an MHC monomer of claim 70, wherein the MHC monomer is loaded with antigen in vitro.

83. A plurality of pMHC multimers of claim 75, wherein the plurality of pMHC multimers and/or MHC multimers comprises one or more pMHC monomer species and/or one or more MHC monomer species, wherein each pMHC monomer species and/or MHC monomer species comprises a different disease-relevant antigen.

84. (canceled)

85. A plurality of high potency receptor-signaling pMHC monomer-nanoparticle conjugates of claim 33.

86. (canceled)

87. A high potency receptor-signaling MHC monomer-nanoparticle conjugate, comprising a nanoparticle core coupled to a plurality of isolated non-peptide tethered pMHC monomers of claim 71, optionally wherein the non-peptide tethered pMHC monomers are coupled to the nanoparticle at a low valency or low density, and wherein the plurality of non-peptide tethered pMHC monomers comprises one or more non-peptide tethered pMHC monomer species, wherein each non-peptide tethered pMHC monomer species comprises a different disease-relevant antigen.

88-91. (canceled)

92. A method for making a high potency receptor-signaling non-peptide tethered pMHC monomer-nanoparticle conjugate comprising: coupling a nanoparticle core to a plurality of isolated non-peptide tethered pMHC monomers of claim 71, optionally wherein the pMHC monomers are coupled to the nanoparticle at a low valency or low density, and wherein the plurality of non-peptide tethered pMHC monomers comprises one or more non-peptide tethered pMHC monomer species, wherein each non-peptide tethered pMHC monomer species comprises a different disease-relevant antigen.

93-105. (canceled)
